(19) United States
(12) Patent Application Publication
(10) Pub. No.: US 2023/0100186 A1
(43) Pub. Date: Mar. 30, 2023

(54) FENTANYL HAPTENS FOR THE PREPARATION OF A FENTANYL VACCINE

(71) Applicants: The Henry M. Jackson Foundation for the Advancement of Military Medicine, Inc., Bethesda, MD (US); The Government of The United States, as represented by The Secretary of The Army, Fort Detrick, MD (US); The United States of America, as represented by the Secretary, Department of Health and Human Serv, Bethesda, MD (US)

(72) Inventors: Oscar Torres, Silver Spring, MD (US); Gary R. Matyas, Silver Spring, MD (US); Kenner C. Rice, Rockville, MD (US); Eric W. Bow, Rockville, MD (US); Agnieszka Sulima, Rockville, MD (US); Arthur E. Jacobson, Rockville, MD (US)

(21) Appl. No.: 17/792,378

(22) PCT Filed: Jan. 13, 2021
(86) PCT No.: PCT/US2021/013300

§ 371 Date: Jul. 12, 2022

Related U.S. Application Data
(60) Provisional application No. 62/960,187, filed on Jan. 13, 2020.

(51) Int. Cl. A61K 39/385; A61K 39/00; A61K 39/39; A61P 25/36

(52) U.S. Cl. CPC A61K 39/385; A61K 39/0013; A61K 39/39; A61P 25/36; A61K 2039/6037; A61K 2039/6081; A61K 2039/55505; A61K 2039/55572; A61K 2039/55555

(57) ABSTRACT

Described is the preparation of novel fentanyl haptens of Formula (1) through (6) and their use in the preparation of effective fentanyl vaccines.

### CROSS-REFERENCE TO RELATED APPLICATIONS

This application claims priority to U.S. Provisional Application No. 62/960,187, filed Jan. 13, 2020, which is incorporated herein by reference for all purposes.

### STATEMENT REGARDING FEDERALLY SPONSORED RESEARCH OR DEVELOPMENT

This invention was made with government support under 1DP1DA034787 and 1UG3DA048351 awarded by the National Institutes of Health. The government has certain rights in the invention.

### BACKGROUND

The dramatic increase in recent years of overdose deaths involving fentanyl represents a significant public health issue. As a result, an urgent need exists to develop a therapy to counteract this trend. One approach has been to prepare a conjugate vaccine for the purpose of eliciting high levels of antibodies with cross-reactivity for various prepared fentanyl haptens (see WO 2017/127390; Pravetoni, M. et al., J. Pharmacol. Exp. Ther. (2019) 368(2):282-291; Bremer, P. T. et al., Angew. Chem. Int. Ed. Engl. (2016) 55(11):3772-3775). Because small-molecule drugs lack immunogenicity on their own, they must be attached to an inmmnunogenic moiety in order for the immune system to recognize and generate antibodies against the target drug. These attempts, however, involve coupling of fentanyl haptens to a carrier protein using carbodiimide chemistry which is difficult to control and which is also prone to oligomerization due to intermolecular reactions of the lysines and the glutamate/aspartate groups present on the surface of the carrier protein. In addition, the structure of the fentanyl hapten is often significantly truncated compared to fentanyl itself (Pravetoni, M. et al., J. Pharmacol. Exp. Ther. (2019) 368(2):282-291).

The present disclosure avoids the aforementioned problems by employing novel fentanyl haptens that contain a thiol linker for facilitating coupling of the hapten to a carrier protein and that induce the formation of antibodies with different specificities for fentanyl derivatives than those reported in the conventional art.

### SUMMARY

Some embodiments provide for a fentanyl hapten of Formula (1), (2) or (3):

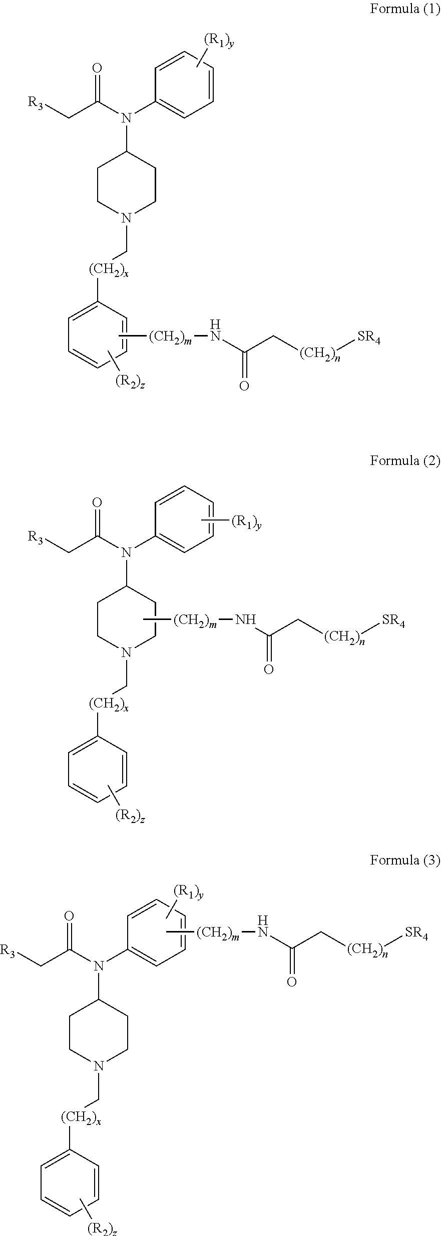

or a pharmaceutically acceptable salt of each thereof.
wherein for Formula (1):
each R₁ and R₂ is independently C₁-C₆ alkyl, C₁-C₆ alkoxy, F, Cl, Br, I, CN, NO₂, NR₅R₆, NR₅COR₆, NR₅CO₂R₇, NR₅SO₂R₇, OR₅, SR₅, SO₂R₂, SO₂NR₅R₆, COR₅, CO₂R₅, CONR₅R₆, CO₂NR₅R₆, cycloalkyl, heterocycloalkyl, aryl or heteroaryl;
R₃ is C₁-C₆ alkyl;
R₄ is H, R₅, NR₅R₆ or C(Ph)₃;
each R₅ is independently H or C₁-C₆ alkyl;
each R₆ is independently H or C₁-C₆ alkyl;
each R₇ is C₁-C₆ alkyl;
m=0, 1, 2, 3, 4, 5 or 6;
n=0, 1, 2, 3, 4, 5 or 6;
x=0, 1, 2, 3, 4, 5 or 6;
y=0, 1, 2, 3, 4 or 5; and
z=0, 1, 2, 3 or 4;
   wherein for Formula (2):
each R₁ and R₂ is independently C₁-C₆ alkyl, C₁-C₆ alkoxy, F, Cl, Br, I, CN, NO₂, NR₅R₆, NR₅COR₆, NR₅CO₂R₇, NR₅SO₂R₇, OR₅, SR₅, SO₂R₂, SO₂NR₅R₆, COR₅, CO₂R₅, CONR₅R₆, CO₂NR₅R₆, cycloalkyl, heterocycloalkyl, aryl or heteroaryl;
R₃ is C₁-C₆ alkyl;
R₄ is H, R₅, COR₅, NR₅R₆ or C(Ph)₃;
each R₅ is independently H or C₁-C₆ alkyl;
each R₆ is independently H or C₁-C₆ alkyl;
each R₇ is C₁-C₆ alkyl;
m=0, 1, 2, 3, 4, 5 or 6;
n=0, 1, 2, 3, 4, 5, or 6;
x=0, 1, 2, 3, 4, 5 or 6;
y=0, 1, 2, 3, 4 or 5; and
z=0, 1, 2, 3, 4 or 5; and
   wherein for Formula (3):
each R₁ and R₂ is independently C₁-C₆ alkyl, C₁-C₆ alkoxy, F, Cl, Br, I, CN, NO₂, NR₅R₆, NR₅COR₆, NR₅CO₂R₇, NR₅SO₂R₂, OR₅, SR₅, SO₂R₂, SO₂NR₅R₆, COR₅, CO₂R₅, CONR₅R₆, CO₂NR₅R₆, cycloalkyl, heterocycloalkyl, aryl or heteroaryl;
R₃ is C₁-C₆ alkyl;
R₄ is H, R₅, COR₅, NR₅R₆ or C(Ph)₃;
each R₅ is independently H or C₁-C₆ alkyl;
each R₆ is independently H or C₁-C₆ alkyl;
each R₇ is C₁-C₆ alkyl;
m=0, 1, 2, 3, 4, 5 or 6;
n=0, 1, 2, 3, 4, 5 or 6;
x=0, 1, 2, 3, 4, 5 or 6;
y=0, 1, 2, 3 or 4; and
z=0, 1, 2, 3, 4 or 5.

An aspect of the disclosure is a fentanyl hapten of Formula (1-i), (2-i) or (3-i):

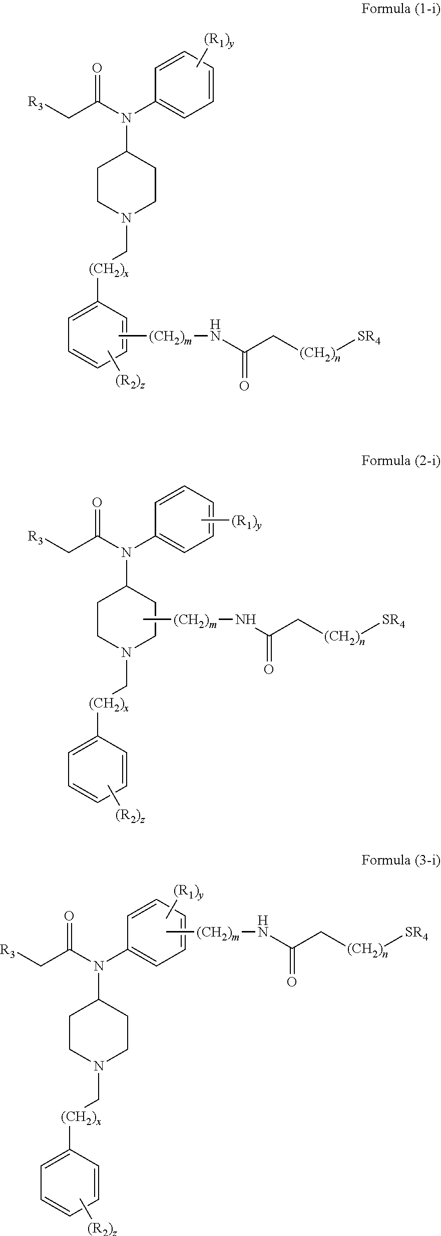

or a pharmaceutically acceptable salt of each thereof,
wherein for Formula (1-i):
each R₁ and R₂ is independently C₁-C₆ alkyl, C₁-C₆ alkoxy, F, Cl, Br, I, CN, NO₂, NR₅R₆, NR₅COR₆, NR₅CO₂R₇, NR₅SO₂R₇, OR₅, SR₅, SO₂R₂, SO₂NR₅R₆, COR₅, CO₂R₅, CONR₅R₆, CO₂NR₅R₆, cycloalkyl, heterocycloalkyl, aryl or heteroaryl, and in the absence of a R₁ or R₂ substituent, a hydrogen atom substituent is present on the phenyl ring;
R₃ is C₁-C₆ alkyl;
R₄ is H, R₅, COR₅, NR₅R₆ or C(Ph)₃;
each R₅ is independently H or C₁-C₆ alkyl;
each R₆ is independently H or C₁-C₆ alkyl;
   each R₇ is C₁-C₆ alkyl;
m=0, 1, 2, 3, 4, 5 or 6;
n=0, 1, 2, 3, 4, 5 or 6;
x=0, 1, 2, 3, 4, 5 or 6;
y=0, 1, 2, 3, 4 or 5; and
z=0, 1, 2, 3 or 4;
   wherein for Formula (2-i):
each R₁ and R₂ is independently C₁-C₆ alkyl, C₁-C₆ alkoxy, F, Cl, Br, I, CN, NO₂, NR₅R₆, NR₅COR₆, NR₅CO₂R₇, NR₅SO₂R₇, OR₅, SR₅, SO₂R₂, SO₂NR₅R₆, COR₅, CO₂R₅, CONR₅R₆, CO₂NR₅R₆, cycloalkyl, heterocycloalkyl, aryl or heteroaryl, and in the absence of a R₁ or R₂ substituent, a hydrogen atom substituent is present on the phenyl ring;
R₃ is C₁-C₆ alkyl;
R₄ is H, R₅, COR₅, NR₅R₆ or C(Ph)₃;
each R₅ is independently H or C₁-C₆ alkyl;
each R₆ is independently H or C₁-C₆ alkyl;
each R₇ is C₁-C₆ alkyl;
m=0, 1, 2, 3, 4, 5 or 6;
n=0, 1, 2, 3, 4, 5, or 6;
x=0, 1, 2, 3, 4, 5 or 6;
y=0, 1, 2, 3, 4 or 5; and
z=0, 1, 2, 3, 4 or 5; and
   wherein for Formula (3-i):
each R₁ and R₂ is independently C₁-C₆ alkyl, C₁-C₆ alkoxy, F, Cl, Br, I, CN, NO₂, NR₅R₆, NR₅COR₆, NR₅CO₂R₇, NR₅SO₂R₂, OR₅, SR₅, SO₂R₇, SO₂NR₅R₆, COR₅, CO₂R₅, CONR₅R₆, CO₂NR₅R₆, cycloalkyl, heterocycloalkyl, aryl or heteroaryl, and in the absence of a R₁ or R₂ substituent, a hydrogen atom substituent is present on the phenyl ring;
R₃ is C₁-C₆ alkyl;
R₄ is H, R₅, COR₅, NR₅R₆ or C(Ph)₃;
each R₅ is independently H or C₁-C₆ alkyl;
each R₆ is independently H or C₁-C₆ alkyl;
each R₇ is C₁-C₆ alkyl;
m=0, 1, 2, 3, 4, 5 or 6;
n=0, 1, 2, 3, 4, 5 or 6;
x=0, 1, 2, 3, 4, 5 or 6;
y=0, 1, 2, 3 or 4; and
z=0, 1, 2, 3, 4 or 5.

### BRIEF DESCRIPTION OF THE DRAWINGS

The following figures are merely illustrative of specific embodiments of the disclosure and are not intended to otherwise limit the full scope of the disclosure as described.

FIG. 1 shows an exemplary approach for preparing the carrier-fentanyl hapten conjugates (i.e. immunoconjugate) of the present disclosure. The carrier protein is first activated by reaction of surface lysine residues on the protein with a maleimide-donating reagent such as, but not limited to, sulfo-GMBS. The activated carrier protein is then reacted with a fentanyl hapten of the present disclosure where the thiol of the hapten reacts in a Michael addition to form a covalent bond to the maleimide moiety, thus resulting in the generation of the carrier-fentanyl hapten conjugate. A specific example of this approach is also shown.

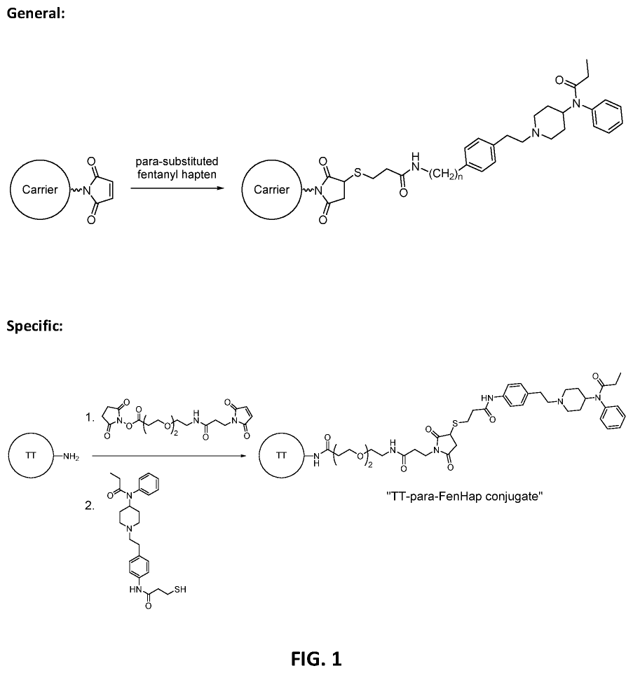

FIG. 2 depicts carrier-fentanyl hapten conjugates for each of the fentanyl hapten compounds of Formula (1), Formula (2) and Formula (3).

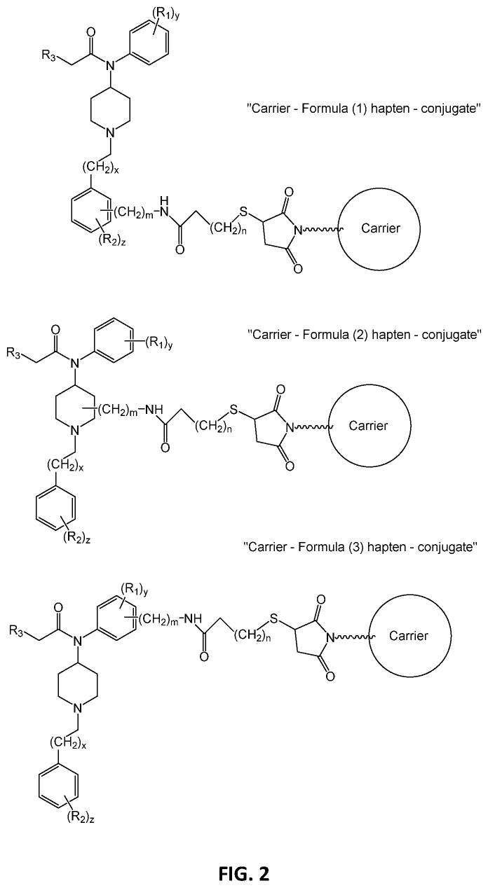

FIG. 3 shows the results of immunizing mice with an exemplary carrier-fentanyl hapten conjugate of the present disclosure (i.e., the π-para-FenHap conjugate shown in FIG. 1) (circles) compared to a control (squares). The control mice and the immunized mice were challenged with increasing doses of fentanyl at week 18 and week 22 post-vaccination. The efficacy of the fentanyl vaccine of the disclosure was assessed by measuring the maximum possible effect (% MPE) in both tail immersion and hot plate assays. The Week 18 challenge demonstrates that mice immunized with the fentanyl vaccine have a lower % MPE compared to the control mice. The Week 22 challenge demonstrates that mice immunized with the fentanyl vaccine have a 26- to 27-fold increase in ED₅₀ (50% effective dose).

FIG. 4 shows that various exemplary carrier-fentanyl hapten conjugates as described herein protected the mice against fentanyl challenge (data shown taken at week 22 post-vaccination).

### DETAILED DESCRIPTION

### Definitions

The term “pharmaceutically acceptable carrier,” as used herein, includes any and all solvents, or a dispersion medium including, but not limited to, water, ethanol, a polyol (for example, glycerol, propylene glycol, and liquid polyethylene glycol, and the like), suitable mixtures thereof, and vegetable oils, coatings, isotonic and absorption delaying agents, liposomes, commercially available cleansers, and the like. Supplementary bioactive ingredients also can be incorporated into such carriers.

The term “hapten,” as used herein, includes peptides, small molecules and modified versions thereof which are used as antigens. Haptens are able to act as recognition sites for the production of specific antibodies but cannot by themselves stimulate the necessary immune response. Haptens can be made immunogenic by coupling them to a suitable carrier molecule, such as a protein, which can be processed by antigen presenting cells and presented to the immune system such that the immune system recognizes the unmodified small molecule. Further, the hapten may be characterized as the specificity-determining portion of the hapten-carrier conjugate that is capable of reacting with an antibody specific to the hapten in its free state.

The term “crosslinker” or “cross-linker” or “linker” or “linking moiety,” as used herein, refers to a moiety having a chemical functionality suitable for attachment of the hapten to a spacer or a carrier. If the conjugation moiety is not suitable for conjugation directly with the carrier moiety, a linker moiety comprising chemical functionality suitable for conjugation with the conjugation moiety and protein moiety can be used. There is a wide range of conventional methods known for linking conjugation moieties to carrier moieties. The length and nature of the linker is such that the hapten is displaced a sufficient distance from the carrier moiety to elicit a suitable antibody response to the hapten in vivo. There are also numerous conventional methods for conjugating polypeptides to carrier moieties and/or linker moieties. Several hetero-bifunctional cross-linkers are known in the art, where the hetero-bifunctional cross-linker contains a functional group which reacts with the free amino groups of the lysine residues of a protein and a functional group which reacts with a cysteine residue or sulfhydryl group present on the hapten, thereby leading to the formation of a thioether linkage. The cysteine residue or sulfhydryl group can be naturally present on the hapten, made available for reaction by reduction, or engineered or attached on the hapten and optionally made available for reaction by reduction. Exemplary commercially available hetero-bifunctional cross-linkers include SM PH, Sulfo-MBS, Sulfo-EMCS, Sulfo-GMBS, Sulfo-SIAB, Sulfo-SMPB, Sulfo-SMCC, SVSB, and SIA.

The term “spacer,” as used herein, refers to the chemical moiety that provides a molecular distance between the hapten and the carrier. In general, any conventional spacer is suitable, which is typically a modified or unmodified carbon chain. An exemplary embodiment, the spacer is one or more polyethylene glycol (PEG) units. PEG is a useful spacer moiety because of its desirable properties of water solubility, high mobility in solution, lack of toxicity and immunogenicity, ready clearance from the body and altered distribution in the body. In exemplary embodiments, the spacer comprises 1 to about 5 consecutive PEG spacers, where the spacer length is from 1 to about 40 PEG units and where the PEG units can be linear or branched. In some embodiments, the PEG spacer has a molecular weight of ≥5 kDa. In some embodiments, the PEG spacer has a molecular weight of about 1 kDA to about 5 kDa.

The term “liposome,” as used herein, includes closed bilayer membranes containing an entrapped aqueous volume and may also be unilamellar vesicles possessing a single membrane bilayer or multilamellar vesicles with multiple membrane bilayers, each separated from the next by an aqueous layer. In the resulting membrane bilayer, the hydrophobic (non-polar) tails of the lipid are oriented toward the center of the bilayer while the hydrophilic (polar) heads orient towards the aqueous phase. Suitable hydrophilic polymers for surrounding the liposomes include PEG, polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxethylacrylate, hydroxymethylcellulose hydroxyethylcellulose, polyethyleneglycol, polyaspartamide and hydrophilic peptide sequences as described in U.S. Pat. Nos. 6,316,024; 6,126,966; 6,056,973; and 6,043,094, all of which are incorporated by reference in their entireties. Liposomes can also be prepared without hydrophilic polymers. Vesicle-forming lipids may be naturally-occurring or synthetic and include phospholipids, such as phosphatidylcholine, phosphatidylethanolamine, phosphatidic acid, phosphatidylserine, phasphatidylglycerol, phosphatidylinositol and sphingomyelin as disclosed in U.S. Pat. Nos. 6,056,973 and 5,874,104. Vesicle-forming lipids also include glycolipids, cerebrosides, or cationic lipids, such as 1,2-dioleyloxy-3-(trimethylamino)propane (DOTAP); N-[1-(2,3,-ditetradecyloxy)propyl]-N,N-dimethyl-N-hydroxyethylammonium bromide (DMRIE); N-[1[(2,3,-dioleyloxy)propyl]-N,N-dimethyl-N-hydroxy ethylammonium bromide (DORIE); N-[1-(2,3-dioleyloxyl)propyl]-N,N,N-trimethylammonium chloride (DOTMA); 3-[N—(N′,N′-dimethylaminoethane) carbamoly]cholesterol (DC-Chol); or dimethyldioctadecylammonium (DDAB) also as disclosed in U.S. Pat. No. 6,056,973. Cholesterol may also be present in the proper range to impart stability to the vesicle as disclosed in U.S. Pat. Nos. 5,916,588 and 5,874,104. Additional liposomal technologies are described in U.S. Pat. Nos. 9,193,739; 6,759,057; 6,406,713; 6,352,716; 6,316,024; 6,294,191; 6,126,966; 6,056,973; 6,043,094; 5,965,156; 5,916,588; 5,874,104; 5,215,680; and 4,684,479, all of which are incorporated herein by reference in their entireties. Army Liposome Formulation (ALF) is composed of a family of suitable anionic liposome-based adjuvants, in which the liposomes contain synthetic phospholipids having dimyristoyl fatty acyl groups, cholesterol and monophosphoryl lipid A (MPLA). ALF may be used in combination with another vaccine adjuvant, such as an aluminum salt, such as aluminum hydroxide (AH), to form ALFA. Liposomes containing the saponin Q21 (ALFQ) have also been combined with AH to form ALFQA.

The term “carrier,” as used herein, refers to a moiety capable of enhancing the immunogenicity of a hapten. Carriers are typically large, slowly metabolized macromolecules such as proteins, polysaccharides, polymeric amino acids, amino acid copolymers and inactive virus particles or attenuated bacteria. Exemplary carrier proteins include, but are not limited to, serum albumins, edestin, keyhole limpet hemocyanin (KLH), recombinant KLH, sheep red blood cells, selected immunoglobulin molecules, thyroglobulin, human serum albumin, ovalbumin, bovine serum albumin (BSA), exotoxins (e.g., Exotoxin A), tetanus toxoid (TT), recombinant mutated TT, recombinant tetanus A chain, recombinant tetanus B chain, diphtheria toxoid (CRM), CRM197, D-lysine, D-glutamic acid, selected members of the LTB family of bacterial toxins, recombinant pox virus subunits, retrovirus nucleoprotein, vesicular stomatitis virus nucleocapsid protein and rabies ribonucleoprotein. A specific carrier is MPER, where MPER is an amino acid sequence of the membrane proximal external region (MPER) of HIV-1 gp41 protein. In an exemplary embodiment, the carrier is embedded in a liposome, with the carrier being of sufficient length to anchor the immunoconjugate complex (e.g., the fentanyl hapten-carrier conjugate) to the liposome surface.

The term “adjuvant,” as used herein, refers to a component administered with a vaccine that helps create a stronger immune response in a subject receiving the vaccine. Aluminum salts are suitable adjuvants and include, but are not limited to, amorphous aluminum hydroxyphosphate sulfate (AAHS), aluminum hydroxide (AH), aluminum phosphate and potassium aluminum sulfate (alum). Liposomes are also suitable adjuvants, especially those containing monophosphoryl lipid A and/or saponin QS21, such as, for example, Army Liposome Formulations (ALF) (liposomes containing monophosphoryl lipid A) and ALFQ (liposomes containing monophosphoryl lipid A and QS21). ALF and ALFQ can also be combined with aluminum salts to yield, for example, ALFA (ALF and aluminum hydroxide) and ALFQA (ALFQ and aluminum hydroxide). Other suitable adjuvants are well-known and described in Vaccine Adjuvants (edited by Derek T, O'Hagan, Humana Press 2000), the contents of which are incorporated by reference.

The term “preservative,” as used herein, includes one or more excipients that prevent contamination (by, e.g., an external fungus or bacteria) of a formulation or composition containing an active ingredient (such as thimerosal, phenol, benzethonium chloride, 2-phenoxy-ethanol).

The term “stabilizer,” as used herein, includes one or more excipients that protect the integrity of the active ingredients during manufacture, storage and transport. Examples are well known and include, but are not limited to, any of various sugars (e.g., lactose, sucrose), gelatin, glycine, 2-phenoxy-ethanol, polysorbate 80 or human or bovine serum albumin.

The term “alkyl,” as used herein, means any straight chain or branched, non-cyclic or cyclic, unsaturated or saturated aliphatic hydrocarbon containing, for example, from 1 to 20 carbon atoms, while the term “lower alkyl” has the same meaning as alkyl but contains from 1 to 5 carbon atoms. The term “higher alkyl” has the same meaning as alkyl but contains from 6 to 20 carbon atoms. Representative saturated straight chain alkyls include, but are not limited to, methyl, ethyl, n-propyl, n-butyl, n-pentyl, n-hexyl, n-heptyl, n-octyl, n-nonyl, and the like; while saturated branched alkyls include, but are not limited to, isopropyl, sec-butyl, isobutyl, tert-butyl, isopentyl, and the like. Cyclic alkyls may be obtained by joining two alkyl groups bound to the same atom or by joining two alkyl groups each bound to adjoining atoms. Representative saturated cyclic alkyls include, but are not limited to, cyclopropyl, cyclobutyl, cyclopentyl, cyclohexyl, and the like; while unsaturated cyclic alkyls include, but are not limited to, cyclopentenyl and cyclohexenyl, and the like. Cyclic alkyls are also referred to herein as “cycloalkyls,” “homocycles” or “homocyclic rings.” Unsaturated alkyls contain at least one double or triple bond between adjacent carbon atoms (referred to as an “alkenyl” or “alkynyl,” respectively). Representative straight chain and branched alkenyls include, but are not limited to, ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include, but are not limited to, acetylenyl, propynyl, 1-butynyl, 2- butynyl, 1- pentynyl, 2-pentynyl, 3-methyl-1-butynyl, and the like.

The term “aryl,” as used herein, refers to any aromatic carbocyclic moiety containing, for example, 5 to 20 carbon atoms such as, but not limited to, phenyl or naphthyl.

The term “arylalkyl” or “aralkyl,” as used herein, refers to any alkyl having at least one alkyl hydrogen atom replaced with an aryl moiety, such as benzyl, but not limited to, (CH₂)₂phenyl, —(CH₂)₃phenyl, —CH(phenyl)₂, and the like.

The term “halogen” or “halo,” as used herein, refers to any fluoro, chloro, bromo, or iodo moiety.

The term “heteroaryl” or “heteroaromatic,” as used herein, refers to any aromatic heterocycle ring of 5 to 20 members and having at least one heteroatom selected from nitrogen, oxygen and sulfur, and containing at least one carbon atom, including, but not limited to, both mono- and bicyclic ring systems. Representative heteroaryls include, but are not limited to, furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, or quinazolinyl.

The term “heteroarylalkyl,” as used herein, refers to any alkyl having at least one alkyl hydrogen atom replaced with a heteroaryl moiety, such as —CHpyridinyl, —CH₂pyrimidinyl, and the like.

The term “heterocycle” or “heterocyclic,” “heterocyclyl” or “heterocyclic ring,” as used herein, refers to any non-aromatic 3- to 7-membered monocyclic or any non-aromatic 7- to 10-membered bicyclic heterocyclic ring which is either saturated or unsaturated and which contains from 1 to 4 heteroatoms independently selected from nitrogen, oxygen and sulfur, and wherein the nitrogen and sulfur heteroatoms may be optionally oxidized, and the nitrogen heteroatom may be optionally quaternized, including bicyclic rings in which any of the above heterocycles are fused to a benzene ring. The heterocycle may be attached via any heteroatom or carbon atom. Heterocycles may include, but are not limited to, morpholinyl, pyrrolidinonyl, pyrrolidinyl, piperidinyl, piperazinyl, imidazolinyl, imidazolidinyl, pyrazolinyl, pyrazolidinyl, 1,4-dioxanyl, dithianyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

The term “heterocycloalkyl,” as used herein, refers to any alkyl having at least one alkyl hydrogen atom replaced with a heterocycle, such as —CH2morpholinyl, and the like.

The term “homocycle” or “cycloalkyl,” as used herein, refers to any saturated or unsaturated (non-aromatic) carbocyclic ring containing from 3-7 carbon atoms, such as, but not limited to, cyclopropane, cyclobutane, cyclopentane, cyclohexane, cycloheptane, cyclohexene, and the like.

The term “alkylamino,” as used herein, refers to at least one alkyl moiety attached through a nitrogen bridge (i.e., —N-(alkyl)N, such as a dialkylamino) including, but not limited to, methylamino, ethylamino, dimethylamino, diethylamino, and the like.

The term “alkyloxy” or “alkoxy,” as used herein, refers to any alkyl moiety attached through an oxygen bridge (i.e., —O-alkyl) such as, but not limited to, methoxy, ethoxy, and the like.

The term “alkylthio,” as used herein, refers to any alkyl moiety attached through a sulfur bridge (i.e., —S— alkyl) such as, but not limited to, methylthio, ethylthio, and the like.

The term “salts,” as used herein, refers to any salt that complexes with identified compounds described herein. Examples of such salts include, but are not limited to, acid addition salts formed with inorganic acids (e.g., hydrochloric acid, hydrobromic acid, sulfuric acid, phosphoric acid, nitric acid, and the like), and salts formed with organic acids such as, but not limited to, acetic acid, oxalic acid, tartaric acid, succinic acid, malic acid, fumaric acid, maleic acid, ascorbic acid, benzoic acid, tannic acid, pamoic acid, alginic acid, polyglutamic, acid, naphthalene sulfonic acid, naphthalene disulfonic acid, and polygalacturonic acid. Salt compounds can also be administered as pharmaceutically acceptable quaternary salts known to a person skilled in the art, which specifically includes the quaternary ammonium salts of the formula —NRR′R″+Z—, wherein R, R′, R″ is independently hydrogen, alkyl, or benzyl, and Z is a counter ion, including, but not limited to, chloride, bromide, iodide, alkoxide, toluenesulfonate, methylsulfonate, sulfonate, phosphate, or carboxylate (such as benzoate, succinate, acetate, glycolate, maleate, malate, fumarate, citrate, tartrate, ascorbate, cinnamoate, mandeloate, and diphenylacetate). Salt compounds can also be administered as pharmaceutically acceptable pyridine cation salts having a substituted or unsubstituted partial formula: wherein Z is a counter ion, including, but not limited to, chloride, bromide, iodide, alkoxide, toluenesulfonate, methylsulfonate, sulfonate, phosphate, or carboxylate (such as benzoate, succinate, acetate, glycolate, maleate, malate, fumarate, citrate, tartrate, ascorbate, cinnamoate, mandeloate, and diphenylacetate).

The term “pharmaceutically acceptable salt” of a given compound refers to salts that retain the biological effectiveness and properties of the given compound, and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, signify that the quantity and/or magnitude of the symptoms in the treated subject is lower than in the untreated subject by any amount or degree that is recognized as clinically relevant by any medically trained personnel. In various exemplary embodiments, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “inhibitory compound,” as used herein, refers to any compound capable of interacting with (i.e., for example, attaching, binding etc.) to a binding partner under conditions such that the binding partner becomes unresponsive to its natural ligands. Inhibitory compounds may include, but are not limited to, small organic molecules, antibodies, and proteins/peptides.

The term “attached,” as used herein, refers to any interaction between a medium (or carrier) and a drug. Attachment may be reversible or irreversible. Such attachment includes, but is not limited to, covalent bonding, ionic bonding, Van der Waals forces or friction, and the like. A drug is attached to a medium (or carrier) if it is impregnated, incorporated, coated, in suspension with, in solution with, mixed with, etc.

The term “drug” or “compound,” as used herein, refers to any pharmacologically active substance capable of being administered which achieves a desired effect. Drugs or compounds can be synthetic or naturally occurring, non-peptide, proteins or peptides, oligonucleotides or nucleotides, polysaccharides or sugars.

The term “administered” or “administering,” as used herein, refers to any method of providing a composition to a patient such that the composition has its intended effect on the patient. An exemplary method of administering is by a direct mechanism such as, local tissue administration (i.e., for example, extravascular placement), oral ingestion, transdermal patch, topical, inhalation, suppository, etc.

The term “patient,” as used herein, is an animal, such as, for example, a mammal, such as, for example, a human that need not be hospitalized. For example, out-patients and persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “subject,” as used herein, refers to a vertebrate, preferably a mammal, more preferably a primate, still more preferably a human. Mammals include, without limitation, humans, primates, wild animals, feral animals, farm animals, sports animals and pets. Examples of non-human mammals include horse, donkey, pig, mouse, rat, hamster, monkey and chicken. The subject may be a juvenile or an adult.

In the present context, the term “therapeutically effective” or “effective amount” indicates that the materials or amount of material is effective to prevent, alleviate, or ameliorate one or more symptoms of a disease or medical condition, and/or to prolong the survival of the subject being treated. The therapeutically effective amount will vary depending on the compound, the disorder or condition and its severity and the age, weight, etc., of the mammal to be treated. For example, an effective amount is an amount sufficient to effectuate a beneficial or desired clinical result.

The term, “immunologically effective amount” refers to an amount of material sufficient to trigger an immunological response in a subject. The effective amounts can be provided all at once in a single administration or in fractional amounts that provide the effective amount in several administrations. The precise determination of what would be considered an effective amount may be based on factors individual to each subject, including their size, age, injury, and/or disease or injury being treated, and amount of time since the injury occurred or the disease began. One skilled in the art will be able to determine the effective amount for a given subject based on these considerations which are routine in the art.

Some embodiments provide for a fentanyl hapten of Formula (1), (2), or (3) or a pharmaceutically acceptable salt thereof. Some embodiments provide for a fentanyl hapten of Formula (1) or a pharmaceutically acceptable salt thereof. Some embodiments provide for a fentanyl hapten of Formula (2) or a pharmaceutically acceptable salt thereof. Some embodiments provide for a fentanyl hapten of Formula (3) or a pharmaceutically acceptable salt thereof.

In an exemplary embodiment, R₃ is methyl (—CH3) for each of Formula (1), (2) and (3).

In an exemplary embodiment, n=1 for each of Formula (1), (2) and (3).

In an exemplary embodiment, x=1 for each of Formula (1), (2) and (3).

In an exemplary embodiment, z=0 for each of Formula (1), (2) and (3).

In an exemplary embodiment, y=0 for each of Formula (1), (2) and (3).

In an exemplary embodiment, R₄ is H for each of Formula (1), (2) and (3). In an exemplary embodiment, R₄ is C(Ph)₃ for each of Formula (1), (2) and (3).

In an exemplary embodiment, R₃ is CH₃, x=1 and n=1 for each of Formula (1), (2) and (3).

In an exemplary embodiment, R₃ is CH₃, R₄ is H, x=1 and n=1 for each of Formula (1), (2) and (3).

In an exemplary embodiment, R₃ is CH₃, R₄ is H, x=1, y=0 and n=1 for each of Formula (1), (2) and (3).

In an exemplary embodiment, R₃ is CH₃, R₄ is H, x=1, y=0, z=0 and n=1 for each of Formula (1), (2) and (3).

In an exemplary embodiment, R₃ is CH₃, R₄ is H, x=1, y=0, z=0, m=0 and n=1 for each of Formula (1), (2) and (3).

In an exemplary embodiment, R₃ is CH₃, R₄ is H, x=1, y=0, z=0, m=1 and n=1 for each of Formula (1), (2) and (3).

In an exemplary embodiment, R₃ is CH₃, R₄ is H or C(Ph)₃, x=1, y=0, and z=0 and the —(CH₂)ₘ—NH—CO—(CH₂)ₙ—SR₄ substituent is in the ortho position on the benzene ring for Formula (1) and Formula (3).

In an exemplary embodiment, R₃ is CH₃, R₄ is H or C(Ph)₃, x=1, y=0, and z=0 and the —(CH₂)ₘ—NH—CO—(CH₂)ₙ—SR₄ substituent is in the meta position on the benzene ring for Formula (1) and Formula (3).

In an exemplary embodiment, R₃ is CH₃, R₄ is H or C(Ph)₃, x=1, y=0, and z=0 and the —(CH₂)ₘ—NH—CO—(CH₂)ₙ—SR₄ substituent is in the para position for Formula (1) and Formula (3).

In an exemplary embodiment, R₃ is CH₃, R₄ is H or C(Ph)₃, x=1, y=0, z=0 and n=1 and the —(CH₂)ₘ—NH—CO—(CH₂)ₙ—SR₄ substituent is in the ortho position on the benzene ring for Formula (1) and Formula (3).

In an exemplary embodiment, R₃ is CH₃, R₄ is H or C(Ph)₃, x=1, y=0, z=0 and n=1 and the —(CH₂)ₘ—NH—CO—(CH₂)ₙ—SR₄ substituent is in the meta position on the benzene ring for Formula (1) and Formula (3).

In an exemplary embodiment, R₃ is CH₃, R₄ is H or C(Ph)₃, x=1, y=0, z=0 and n=1 and the —(CH₂)ₘ—NH—CO—(CH₂)ₙ—SR₄ substituent is in the para position for Formula (1) and Formula (3).

In an exemplary embodiment, R₃ is CH₃, R₄ is H, x=1, y=0, z=0 and n=1 and the —(CH₂)ₘ—NH—CO—(CH₂)ₙ—SR₄ substituent is in the ortho position on the benzene ring for Formula (1) and Formula (3). In an exemplary embodiment, R₃ is CH₃, R₄ is H, x=1, y=0, z=0 and n=1 and the —(CH₂)ₘ—NH—CO—(CH₂)ₙ—SR₄ substituent is in the ortho position on the benzene ring for Formula (1). In an exemplary embodiment, R₃ is CH₃, R₄ is H, x=1, y=0, z=0 and n=1 and the —(CH₂)ₘ—NH—CO—(CH₂)ₙ—SR₄ substituent is in the ortho position on the benzene ring for Formula (3).

In an exemplary embodiment, R₃ is CH₃, R₄ is H, x=1, y=0, z=0 and n=1 and the —(CH₂)ₘ—NH—CO—(CH₂)ₙ—SR₄ substituent is in the meta position on the benzene ring for Formula (1) and Formula (3). In an exemplary embodiment, R₃ is CH₃, R₄ is H, x=1, y=0, z=0 and n=1 and the —(CH₂)ₘ—NH—CO—(CH₂)ₙ—SR₄ substituent is in the meta position on the benzene ring for Formula (1). In an exemplary embodiment, R₃ is CH₃, R₄ is H, x=1, y=0, z=0 and n=1 and the —(CH₂)ₘ—NH—CO—(CH₂)ₙ—SR₄ substituent is in the meta position on the benzene ring for Formula (3).

In an exemplary embodiment, R₃ is CH₃, R₄ is H, x=1, y=0, z=0 and n=1 and the —(CH₂)ₘ—NH—CO—(CH₂)ₙ—SR₄ substituent is in the para position for Formula (1) and Formula (3). In an exemplary embodiment, R₃ is CH₃, R₄ is H, x=1, y=0, z=0 and n=1 and the —(CH₂)ₘ—NH—CO—(CH₂)ₙ—SR₄ substituent is in the para position for Formula (1). In an exemplary embodiment, R₃ is CH₃, R₄ is H, x=1, y=0, z=0 and n=1 and the —(CH₂)ₘ—NH—CO—(CH₂)ₙ—SR₄ substituent is in the para position for Formula (3).

The bond that connects the —(CH₂)ₘ—NH—CO—(CH₂)ₙ—SR₄ substituent to the piperidine ring in Formula (2) is intended to be racemic as shown. In an exemplary embodiment, the bond has hashed ( [symbol] ) stereochemistry. In another exemplary embodiment, the bond has wedged ( [symbol] ) stereochemistry.

Another aspect of the disclosure is an immunoconjugate comprising a fentanyl hapten of Formula (1), (2) or (3).

In an exemplary embodiment, the immunoconjugate comprises a carrier and a fentanyl hapten of Formula (1), (2) or (3), wherein the hapten is covalently linked directly or indirectly to the carrier.

In some embodiments, the immunoconjugate comprises a carrier and the fentanyl hapten of Formula (1), (2) or (3) as described herein, wherein the fentanyl hapten is covalently linked to the carrier.

In some embodiments, the immunoconjugate comprises a carrier attached to a linking moiety, and a fentanyl hapten of Formula (1), (2) or (3) as described herein attached to the linking moiety.

In an exemplary embodiment, the carrier is selected from the group consisting of tetanus toxoid (TT), CRM197, diphtheria toxoid, recombinant deactivated tetanus toxoid, recombinant tetanus A chain, recombinant tetanus B chain, exotoxin A, keyhole limpet hemocyanin (KLH) and recombinant KLH. In an exemplary embodiment, the carrier is selected from the group consisting of bovine serum albumin (BSA), tetanus toxoid (TT), CRM197, diphtheria toxoid, recombinant deactivated tetanus toxoid, recombinant tetanus A chain, recombinant tetanus B chain, exotoxin A, keyhole limpet hemocyanin (KLH) and recombinant KLH.

In a particular embodiment, the carrier is bovine serum albumin.

In a particular embodiment, the carrier is tetanus toxoid.

Another aspect of the disclosure is a composition comprising an immunologically effective amount of the immunoconjugate and a physiologically acceptable vehicle.

In an exemplary embodiment, the composition further comprises an adjuvant.

In exemplary embodiment, the adjuvant is selected from the group consisting of an ALF liposome, ALFA, ALFQ, ALFQA, aluminium hydroxide, aluminium phosphate, alum and monophosphoryl lipid A containing adjuvants.

In an exemplary embodiment, the adjuvant is an Army Liposome Formulation (ALF).

In an exemplary embodiment, the ALF is combined with an aluminum salt.

In an exemplary embodiment, the adjuvant is ALF combined with aluminum hydroxide as a second adjuvant (ALFA).

In an exemplary embodiment, the adjuvant is ALFQ, where the liposomes contain the saponin QS21.

In an exemplary embodiment, the adjuvant is ALFQ combined with aluminum hydroxide as a second adjuvant (ALFQA).

In an exemplary embodiment, the adjuvant is selected from the group consisting of ALF, ALFA, ALFQ and ALFQA, aluminium hydroxide, aluminium phosphate, alum and monophosphoryl lipid A.

In an exemplary embodiment, the immunoconjugate is embedded, associated with or attached to the adjuvant.

In an exemplary embodiment, the composition further comprises a preservative.

In an exemplary embodiment, the composition further comprises a stabilizer.

Another aspect of the disclosure is a method for inducing an anti-fentanyl immune response in a subject, comprising immunizing the subject with an immunologically effective amount of a composition comprising an immunoconjugate as described herein and a physiologically acceptable vehicle.

Another aspect of the disclosure is a method for inducing an anti-fentanyl immune response in a subject, comprising immunizing the subject with an immunologically effective amount of a composition comprising an immunologically effective amount of the immunoconjugate as described and a physiologically acceptable vehicle.

In some embodiments, the immunoconjugate comprises a carrier. In some embodiments, the immunoconjugate comprises a carrier, wherein the carrier is tetanus toxoid.

Another aspect of the disclosure is a method for inducing an anti-fentanyl immune response without inducing an immune response to a carrier moiety in a subject, comprising immunizing the subject with an immunologically effective amount of the composition as described herein.

Another aspect of the disclosure is a method for inducing an anti-fentanyl immune response without also inducing in a subject an immune response to the carrier as described, comprising immunizing the subject with an immunologically effective amount of the composition as described herein, wherein the immunoconjugate is embedded, associated with or attached to the adjuvant.

Another aspect of the disclosure is an antibody that binds the immunoconjugate.

In an exemplary embodiment, the antibody that binds the immunoconjugate binds fentanyl.

Another aspect of the disclosure is a composition comprising the antibody and a physiologically acceptable vehicle.

Another aspect of the disclosure is a vaccine composition comprising the immunoconjugate as described.

In an exemplary embodiment, the vaccine composition further comprises an adjuvant.

In an exemplary embodiment, the adjuvant in the vaccine composition is selected from the group consisting of ALF, ALFA, ALFQ and ALFQA. In some embodiments, the adjuvant is selected from the group consisting of an ALF liposome, ALFA, ALFQ, ALFQA, aluminium hydroxide, aluminium phosphate, alum and monophosphoryl lipid A containing adjuvants.

In an exemplary embodiment, the hapten in the immunoconjugate is covalently linked to the carrier through a linking moiety.

In an exemplary embodiment, the linking moiety is a NHS-maleimide heterobifunctional crosslinker. In some embodiments, the linking moiety is NHS-(PEG)ₙ-maleimide (i.e. SM(PEG)ₙ), wherein n is 2, 4, 6, 8, 12, or 24.

In some embodiments, the fentanyl hapten is covalently linked to the carrier through a linking moiety, where the linking moiety is a NHS-maleimide crosslinker.

In an exemplary embodiment, the vaccine composition further comprises a preservative.

In an exemplary embodiment, the vaccine composition further comprises a stabilizer.

Another aspect of the disclosure is a composition comprising an immunologically effective amount of an immunoconjugate comprising a hapten of Formula (1), (2) or (3) and a physiologically acceptable vehicle.

In an exemplary embodiment, the composition comprising an immunologically effective amount of an immunoconjugate comprising a fentanyl hapten of Formula (1), (2) or (3) and a physiologically acceptable vehicle, further comprises an adjuvant.

In an exemplary embodiment, the composition comprising an immunologically effective amount of an immunoconjugate comprising a fentanyl hapten of Formula (1), (2) or (3) and a carrier, and a physiologically acceptable vehicle, further comprises an adjuvant.

Another aspect of the disclosure is a kit comprising one or more of a fentanyl hapten as described herein or immunoconjugate (such as in the form of a fentanyl hapten-carrier conjugate), an adjuvant, a device for administering the fentanyl hapten (such as in the form of a vaccine) and instructions (such as for administering the vaccine).

In an exemplary embodiment, provided herein is a fentanyl hapten of Formula (1A):

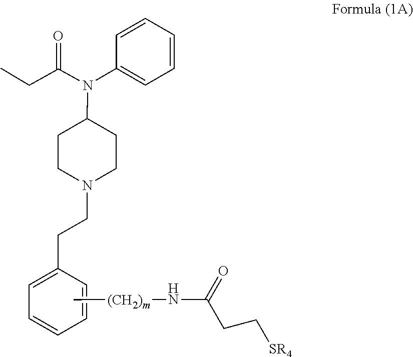

or a pharmaceutically acceptable salt thereof, wherein R₄ is H or C(Ph)₃ and m=0, 1, 2, 3, 4, 5 or 6.

In an exemplary embodiment, provided herein is a fentanyl hapten of Formula (2A):

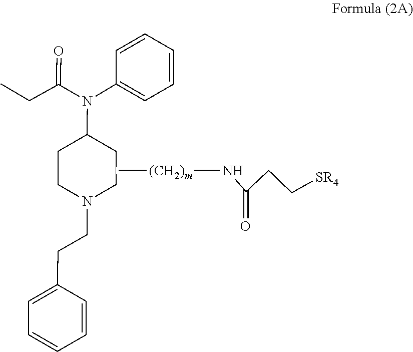

or a pharmaceutically acceptable salt thereof, wherein R₄ is H or C(Ph)₃ and m=0, 1, 2, 3, 4, 5 or 6.

In an exemplary embodiment, provided herein is a fentanyl hapten of Formula (3A):

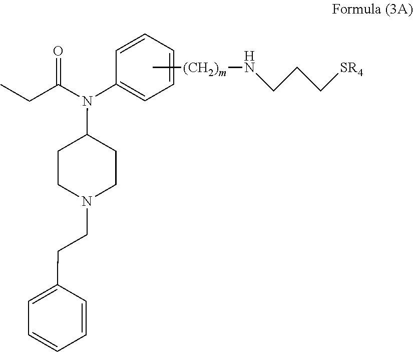

or a pharmaceutically acceptable salt thereof, wherein R₄ is H or C(Ph)₃ and m=0, 1, 2, 3, 4, 5 or 6.

In an exemplary embodiment, the fentanyl hapten of Formula (1) is a fentanyl hapten of Formula (1A-i) where m=0, 1, 2, 3, 4, 5 or 6:

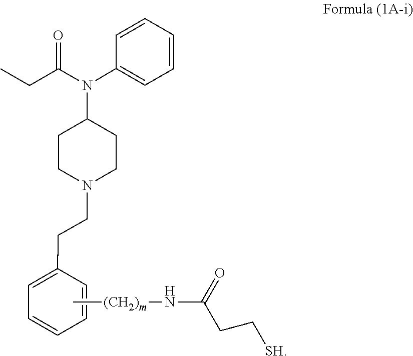

In an exemplary embodiment, the fentanyl hapten of Formula (2) is a fentanyl hapten of Formula (2A-i) where m=0, 1, 2, 3, 4, 5 or 6:

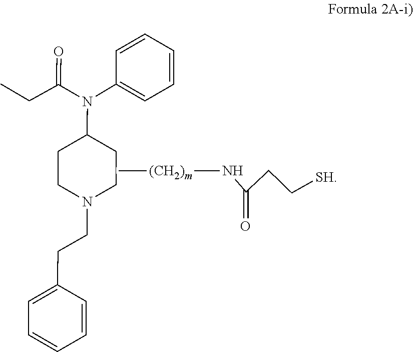

In an exemplary embodiment, the fentanyl hapten of Formula (3) is a fentanyl hapten of Formula (3A-i) where m=0, 1, 2, 3, 4, 5 or 6:

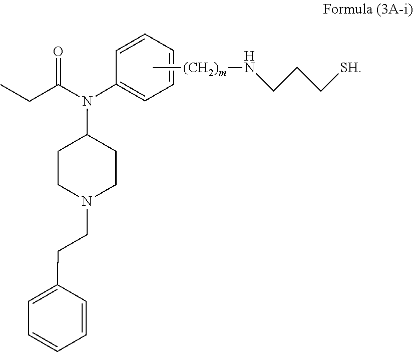

In a particular embodiment, the fentanyl hapten of Formula (1) is 3-mercapto-N-(4-(2-(4-(N-phenylpropionamido)piperidin-1-yl)ethyl)phenyl)propanamide (also referred to herein as “para-AmFenHap” or “para-AmFentanylHap”:

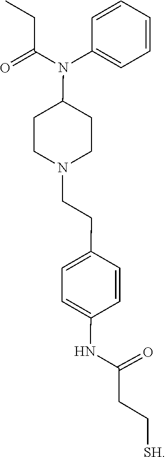 [SMILES: CCC(=O)N(c1ccccc1)C1CCN(CCc2ccc(NC(=O)CCS)cc2)CC1]

In another particular embodiment, the fentanyl hapten of Formula (1) is 3-mercapto-N-(3-(2-(4-(N-phenylpropionamido)piperidin-1-yl)ethyl)phenyl)propanamide:

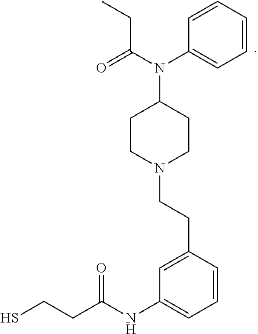 [SMILES: CCC(=O)N(c1ccccc1)C1CCN(CCc2cccc(NC(=O)CCS)c2)CC1]

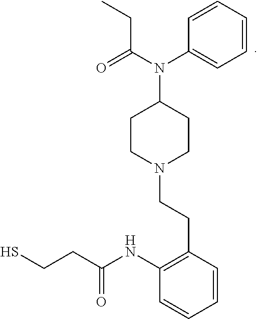 [SMILES: CCC(=O)N(c1ccccc1)C1CCN(CCc2ccccc2NC(=O)CCS)CC1]

In a particular embodiment, the fentanyl hapten of Formula (2) is 3-mercapto-N-(((3R)-1-phenethyl-4-(N-phenylpropionamido)piperidin-3-yl)methyl)propanamide:

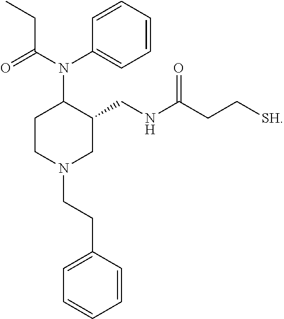 [SMILES: CCC(=O)N(c1ccccc1)C1CCN(CCc2ccccc2)C[C@H]1CNC(=O)CCS]

Another aspect of the disclosure is a fentanyl hapten of Formula (4), (5) or (6)

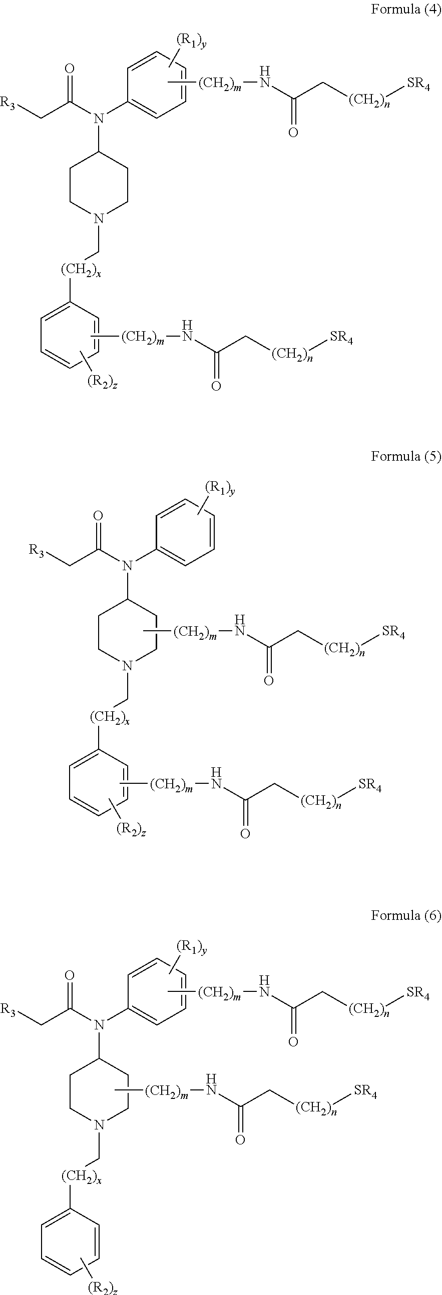

or a pharmaceutically acceptable salt of each thereof,
wherein for Formula (4):
each R₁ and R₂ is independently C₁-C₆ alkyl, C₁-C₆ alkoxy, F, Cl, Br, I, CN, NO₂, NR₅R₆, NR₅COR₆, NR₅CO₂R₇, NR₅SO₂R₇, OR₅, SR₅, SO₂R₂, SO₂NR₅R₆, COR₅, CO₂R₅, CONR₅R₆, CO₂NR₅R₆, cycloalkyl, heterocycloalkyl, aryl or heteroaryl;
R₃ is C₁-C₆ alkyl;
each R₄ is independently H, R₅, COR₅, NR₅R₆ or C(Ph)₃;
each R₅ is independently H or C₁-C₆ alkyl;
each R₆ is independently H or C₁-C₆ alkyl;
each R₇ is C₁-C₆ alkyl;
m=0, 1, 2, 3, 4, 5 or 6;
n=0, 1, 2, 3, 4, 5, or 6;
x=0, 1, 2, 3, 4, 5 or 6;
y=0, 1, 2, 3 or 4; and
z=0, 1, 2, 3 or 4;
   wherein for Formula (5):
each R₁ and R₂ is independently C₁-C₆ alkyl, C₁-C₆ alkoxy, F, Cl, Br, I, CN, NO₂, NR₅R₆, NR₅COR₆, NR₅CO₂R₇, NR₅SO₂R₇, OR₅, SR₅, SO₂R₂, SO₂NR₅R₆, COR₅, CO₂R₅, CONR₅R₆, CO₂NR₅R₆, cycloalkyl, heterocycloalkyl, aryl or heteroaryl;
R₃ is C₁-C₆ alkyl;
each R₄ is independently H, R₅, COR₅, NR₅R₆ or C(Ph)₃;
each R₅ is independently H or C₁-C₆ alkyl;
each R₆ is independently H or C₁-C₆ alkyl;
each R₇ is C₁-C₆ alkyl;
m=0, 1, 2, 3, 4, 5 or 6;
n=0, 1, 2, 3, 4, 5, or 6;
x=0, 1, 2, 3, 4, 5 or 6;
y=0, 1, 2, 3, 4 or 5; and
z=0, 1, 2, 3 or 4; and
   wherein for Formula (6):
each R₁ and R₂ is independently C₁-C₆ alkyl, C₁-C₆ alkoxy, F, Cl, Br, I, CN, NO₂, NR₅R₆, NR₅COR₆, NR₅CO₂R₇, NR₅SO₂R₂, OR₅, SR₅, SO₂R₇, SO₂NR₅R₆, COR₅, CO₂R₅, CONR₅R₆, CO₂NR₅R₆, cycloalkyl, heterocycloalkyl, aryl or heteroaryl;
R₃ is C₁-C₆ alkyl;
each R₄ is independently H, R₅, COR₅, NR₅R₆ or C(Ph)₃;
each R₅ is independently H or C₁-C₆ alkyl;
each R₆ is independently H or C₁-C₆ alkyl;
each R₇ is C₁-C₆ alkyl;
m=0, 1, 2, 3, 4, 5 or 6;
n=0, 1, 2, 3, 4, 5, or 6;
x=0, 1, 2, 3, 4, 5 or 6;
y=0, 1, 2, 3 or 4; and
z=0, 1, 2, 3, 4 or 5.

In an exemplary embodiment, R₃ is CH₃ for each of Formula (4), (5) and (6).

In an exemplary embodiment, each R₄ is independently H or C(Ph)₃ for each of Formula (4), (5) and (6). In an exemplary embodiment, each R₄ is independently H for each of Formula (4), (5) and (6).

The bond that connects the —(CH₂)ₘ—NH—CO—(CH₂)ₙ—SR₄ substituent to the piperidine ring in Formula (5) and Formula (6) is intended to be racemic as shown. In an exemplary embodiment, the bond has hashed ( [symbol] ) stereochemistry. In another exemplary embodiment, the bond has wedged ( [symbol] ) stereochemistry.

Another aspect of the disclosure is an immunoconjugate comprising a fentanyl hapten of Formula (4), (5) or (6).

In an exemplary embodiment, the immunoconjugate comprises a carrier and a fentanyl hapten of Formula (4), (5) or (6), wherein the hapten is covalently linked directly or indirectly to the carrier.

In some embodiments, the immunoconjugate comprises a carrier and the fentanyl hapten of Formula (4), (5) or (6) as described herein, wherein the fentanyl hapten is covalently linked to the carrier.

In some embodiments, the immunoconjugate comprises a carrier attached to a linking moiety, and a fentanyl hapten of Formula (4), (5) or (6) as described herein attached to the linking moiety.

In an exemplary embodiment, the carrier is selected from the group consisting of tetanus toxoid (TT), CRM197, diphtheria toxoid, recombinant deactivated tetanus toxoid, recombinant tetanus A chain, recombinant tetanus B chain, exotoxin A, keyhole limpet hemocyanin (KLH) and recombinant KLH. In a particular embodiment, the carrier is tetanus toxoid. In an exemplary embodiment, the carrier is selected from the group consisting of bovine serum albumin (BSA), tetanus toxoid (TT), CRM197, diphtheria toxoid, recombinant deactivated tetanus toxoid, recombinant tetanus A chain, recombinant tetanus B chain, exotoxin A, keyhole limpet hemocyanin (KLH) and recombinant KLH.

Another aspect of the disclosure is a composition comprising an immunologically effective amount of the immunoconjugate and a physiologically acceptable vehicle.

In an exemplary embodiment, the composition further comprises an adjuvant.

In an exemplary embodiment, the adjuvant is an Army Liposome Formulation (ALF).

In an exemplary embodiment, the ALF is combined with an aluminum salt.

In an exemplary embodiment, the aluminum salt is aluminum hydroxide.

In an exemplary embodiment, the adjuvant is selected from the group consisting of ALF, ALFA, ALFQ and ALFQA, aluminium hydroxide, aluminium phosphate, alum and monophosphoryl lipid A.

In an exemplary embodiment, the immunoconjugate is embedded, associated with or attached to the adjuvant.

In an exemplary embodiment, the composition further comprises a preservative.

In an exemplary embodiment, the composition further comprises a stabilizer.

Another aspect of the disclosure is a method for inducing an anti-fentanyl immune response in a subject, comprising immunizing the subject with an immunologically effective amount of a composition comprising an immunoconjugate as described herein and a physiologically acceptable vehicle.

Another aspect of the disclosure is a method for inducing an anti-fentanyl immune response in a subject, comprising immunizing the subject with an immunologically effective amount of a composition comprising an immunologically effective amount of the immunoconjugate as described and a physiologically acceptable vehicle.

Another aspect of the disclosure is a method for inducing an anti-fentanyl immune response without inducing an immune response to a carrier moiety in a subject, comprising immunizing the subject with an immunologically effective amount of the composition as described herein.

Another aspect of the disclosure is a method for inducing an anti-fentanyl immune response without also inducing in a subject an immune response to the carrier as described, comprising immunizing the subject with an immunologically effective amount of the composition, wherein the immunoconjugate is embedded, associated with or attached to the adjuvant.

Another aspect of the disclosure is an antibody that binds the immunoconjugate.

In an exemplary embodiment, the antibody that binds the immunoconjugate binds fentanyl.

Another aspect of the disclosure is a composition comprising the antibody and a physiologically acceptable vehicle.

Another aspect of the disclosure is a vaccine composition comprising the immunoconjugate as described.

In an exemplary embodiment, the vaccine composition further comprises an adjuvant.

In an exemplary embodiment, the adjuvant in the vaccine composition is selected from the group consisting of ALF, ALFA, ALFQ and ALFQA.

In an exemplary embodiment, the hapten in the immunoconjugate is covalently linked to the carrier through a linking moiety.

In an exemplary embodiment, the linking moiety is a NHS-maleimide crosslinker.

Another aspect of the disclosure is a composition comprising an immunologically effective amount of an immunoconjugate comprising a fentanyl hapten of Formula (4), (5) or (6) and a physiologically acceptable vehicle.

In an exemplary embodiment, the composition comprising an immunologically effective amount of an immunoconjugate comprising a fentanyl hapten of Formula (4), (5) or (6) and a physiologically acceptable vehicle, further comprises an adjuvant.

In an exemplary embodiment, provided herein is a fentanyl hapten of Formula (4A):

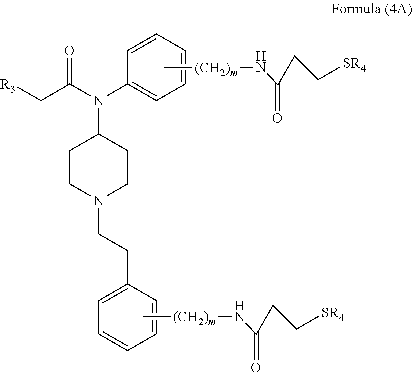

or a pharmaceutically acceptable salt thereof, wherein
R₃ is C₁-C₆ alkyl;
each R₄ is independently H or C(Ph)₃; and
each m is independently 0, 1, 2, 3, 4, 5 or 6.

In an exemplary embodiment, provided herein is a fentanyl hapten of Formula (5A):

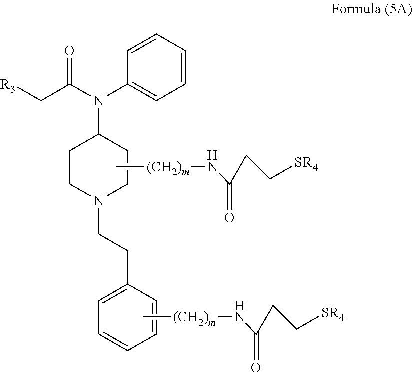

or a pharmaceutically acceptable salt thereof, wherein
R₃ is C₁-C₆ alkyl;
each R₄ is independently H or C(Ph)₃; and
each m is independently 0, 1, 2, 3, 4, 5 or 6.

In an exemplary embodiment, provided herein is a fentanyl hapten of Formula (6A):

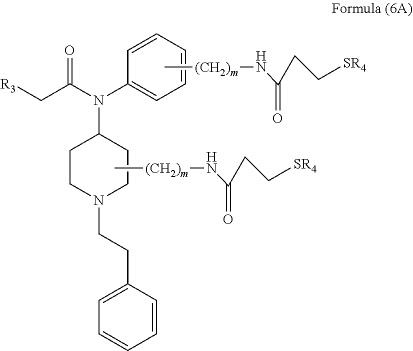

or a pharmaceutically acceptable salt thereof, wherein
R₃ is C₁-C₆ alkyl;
each R₄ is independently H or C(Ph)₃; and
each m is independently 0, 1, 2, 3, 4, 5 or 6.

In an exemplary embodiment, the fentanyl hapten of Formula (4) is a fentanyl hapten of Formula (4A-i) where each m is independently 0, 1, 2, 3, 4, 5 or 6:

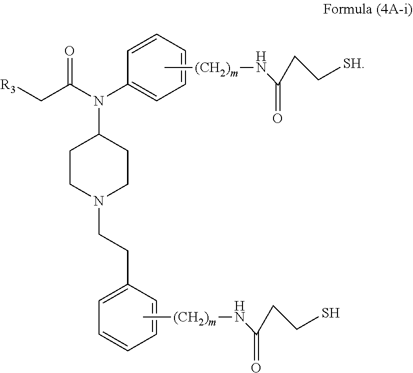

In an exemplary embodiment, the fentanyl hapten of Formula (5) is a fentanyl hapten of Formula (5A-i) where each m is independently 0, 1, 2, 3, 4, 5 or 6:

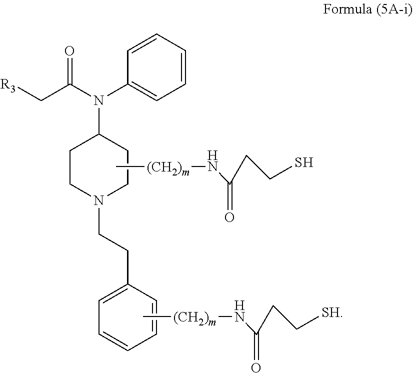

In an exemplary embodiment, the fentanyl hapten of Formula (6) is a fentanyl hapten of Formula (6A-i) where each m is independently 0, 1, 2, 3, 4, 5 or 6:

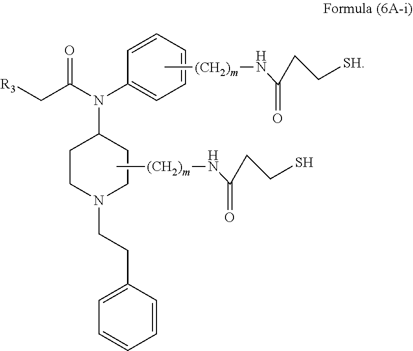

The fentanyl hapten compounds described herein are designed for facile conjugation to a carrier to yield a fentanyl hapten-carrier conjugate, which is then optionally combined with one or more adjuvants to provide a fentanyl vaccine for use in immunization. The antibodies induced by administration of the fentanyl vaccine can bind to fentanyl and fentanyl analogs, including, but not limited to, carfentanil, acryl fentanyl, remifentanyl, alfentanil, lofentanil, sufentanil and trefentanil. Other non-limiting examples of fentanyl analogs include acetyl fentanyl, butyryl fentanyl, o-fluorofentany, p-fluorofentanyl, p-fluorobutyryl fentanyl, p-fluoroisobutyryl fentanyl, furanyl fentanyl, beta-hydroxythiofentanyl, beta-hydroxyfentanyl, isobutyrylfentanyl, 3-methylfentanyl, alpha-methylfentanyl, 4-methoxy-butyryl fentanyl, octfentanil, valeryl fentanyl, and tolyfentanyl.

When fentanyl is injected into the blood of a subject, these antibodies bind the fentanyl and prevent it from crossing the blood-brain barrier. This prevention is of particular importance because fentanyl is known to bind to the μ-receptor in the brain, leading to euphoria, respiratory depression and potentially death. Thus, a vaccine prepared from the fentanyl haptens of the disclosure reduces/prevents fentanyl-related overdoses. In an exemplary embodiment of the disclosure, such a fentanyl vaccine (prepared from the fentanyl haptens described herein) can be used in combination with conventional therapies (e.g., buprenorphine and/or naltrexone) employed to prevent drug overdoses.

In an exemplary embodiment, the disclosure provides a pharmaceutical composition comprising at least one pharmaceutically acceptable carrier or a physiologically acceptable vehicle, in addition to one or more of the compounds described herein (fentanyl hapten or immunoconjugate as described herein). The composition can be present in any suitable form for the desired route of administration. Where the composition is to be administered orally, any suitable orally deliverable dosage form can be used, including, without limitation, tablets, capsules (solid or liquid filled), powders, granules, syrups and other liquids, elixirs, inhalants, troches, lozenges and solutions. Injectable compositions or i.v. infusions are also provided in the form of solutions, suspensions, and emulsions.

The fentanyl hapten compounds of the disclosure or an immunoconjugate as described herein can be formulated as described herein and are suitable for administration to a subject in any number of ways. For example, a therapeutically effective amount of a fentanyl hapten-carrier conjugate as described herein depends upon the amounts and types of additional components (e.g., excipients and/or adjuvants) present in the composition (e.g., a vaccine) to be administered to a subject in need thereof and the route by which the composition is to be administered. In an exemplary embodiment, the route of administration is any one of oral, transcutaneous (skin), subcutaneous, intradermal, intrapertenial, intramuscular and intranasal. In a particular embodiment, the route of administration is intramuscular.

In an exemplary embodiment, the fentanyl haptens of the disclosure are administered in a vaccine as a fentanyl hapten-carrier conjugate, where the carrier is tetanus toxoid or other immunogenic carrier that has an acceptable safety profile and where the amount of the fentanyl hapten-carrier conjugate present in each vaccine dose is selected as the amount which induces an immunoprotective response without significant, adverse side effects. In an exemplary embodiment, a single dose contains 1 to 1000 μg of the fentanyl hapten-carrier conjugate where the fentanyl hapten-carrier conjugate contains 1 to 1000 haptens. These ranges include all values in-between such as, but not limited to, 10 to 800 μg, such as 25 to 600 μg, such as 50 to 500 μg, such as 60 to 350 μg, such as 70 to 300 μg, such as 80 to 200 μg, such as 100 to 200 μg of the fentanyl hapten-carrier conjugate; and 5 to 850, such as 15 to 700, such as 20 to 500, such as 20 to 350, such as 30 to 200, such as 35 to 150, such as 40 to 100, such as 50 to 75 haptens.

The frequency with which the fentanyl haptens are administered to a subject varies depending on several factors, such as the subject's age, condition, sex and other variables which can be adjusted by one of ordinary skill in the art. In an exemplary embodiment, the fentanyl haptens are present in the form of a fentanyl hapten-carrier conjugate and are administered as a vaccine at least once every 3 years, such as at least once every 2 years, such as at least once every 1 year, such as at least once every 6 months, such as at least once every 3 months, such as at least every 2 months, such as at least every month, such as at least every two weeks. The dosage frequency is largely determined by the response observed in the subject with the objective being sufficient frequency to induce an optimal response to the vaccine by the subject's immune system but not frequent enough to induce immune suppression. In an exemplary embodiment, multiple vaccine doses are administered over several weeks to induce high sustained antibody titers. The production of antibodies can be monitored by using conventional techniques, such as ELISA, radioimmunoassay, surface plasma resonance and Western blotting methods.

In an exemplary embodiment, a kit may be employed to store one or more doses of adjuvant compositions and/or one or more doses of compositions containing the fentanyl hapten-carrier conjugate for administration. A kit may also contain instructions for methods for administration, which typically include methods for determining the condition of the subject, the proper dosage amount and the appropriate method for administering the composition. Instructions may also include guidelines for monitoring the subject over the course of the treatment. Kits may also contain devices for administration of the compositions. Exemplary devices include, but are not limited to, a hypodermic or intravenous needle, a catheter, a needle-less injection device, an aerosolizer, an inhaler or nebulizer or atomizer or microspray device, and a liquid dispenser, such as an eyedropper.

Suitable oral compositions in accordance with the disclosure include, without limitation, tablets, troches, lozenges, aqueous or oily suspensions, dispersible powders or granules, emulsion, hard or soft capsules, syrups or elixirs. Inventive compositions suitable for oral use may be prepared according to any method known to the art for the manufacture of pharmaceutical compositions. For example, liquid formulations of the compounds can contain one or more agents selected from the group consisting of sweetening agents, flavoring agents, coloring agents and preserving agents in order to provide pharmaceutically elegant and palatable preparations of the active agents.

For tablet compositions, typical non-toxic pharmaceutically acceptable excipients include, without limitation, inert diluents such as calcium carbonate, sodium carbonate, lactose, calcium phosphate or sodium phosphate; granulating and disintegrating agents such as, for example, corn starch, or alginic acid; binding agents such as, for example, starch, gelatin or lubricating agents such as, for example, magnesium stearate, stearic acid or talc. The tablets may be uncoated or, alternatively, they may be coated by known coating techniques to delay disintegration and absorption in the gastrointestinal tract and thereby to provide a sustained therapeutic action over a desired time period. For example, a time delay material such as glyceryl monostearate or glyceryl distearate may be employed.

Formulations for oral use may also be presented as hard gelatin capsules wherein the active ingredient is mixed with an inert solid diluent such as, for example, calcium carbonate, calcium phosphate or kaolin, or as soft gelatin capsules wherein the active ingredient is mixed with water or an oil medium such as, for example peanut oil, liquid paraffin or olive oil.

For aqueous suspensions, the compound is admixed with excipients suitable for maintaining a stable suspension. Examples of such excipients include, without limitation, sodium carboxymethylcellulose, methylcellulose, hydropropylmethylcellulose, sodium alginate, polyvinylpyrrolidone, gum tragacanth and gum acacia.

Oral suspensions can also contain dispersing or wetting agents, such as naturally-occurring phosphatide such as, for example, lecithin, or condensation products of an alkylene oxide with fatty acids such as, for example, polyoxyethylene stearate, or condensation products of ethylene oxide with long chain aliphatic alcohols such as, for example, heptadecaethyleneoxycetanol, or condensation products of ethylene oxide with partial esters derived from fatty acids and a hexitol such as, for example, polyoxyethylene sorbitol monooleate, or condensation products of ethylene oxide with partial esters derived from fatty acids and hexitol anhydrides such as, for example, polyethylene sorbitan monooleate. The aqueous suspensions may also contain one or more preservatives such as, for example, ethyl or n-propyl p-hydroxybenzoate, one or more coloring agents, one or more flavoring agents, and one or more sweetening agents such as sucrose or saccharin.

Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water can provide the active ingredient in admixture with a dispersing or wetting agent, suspending agent and one or more preservatives. Suitable dispersing or wetting agents and suspending agents are exemplified by those already mentioned above. Additional excipients such as, for example, sweetening, flavoring and coloring agents, may also be present.

Syrups and elixirs may be formulated with sweetening agents such as, for example, glycerol, propylene glycol, sorbitol or sucrose. Such formulations may also contain a demulcent, a preservative, and flavoring and coloring agents.

Compositions for parenteral administrations are formulated in a sterile medium suitable for intravenous, intramuscular or intrathecal delivery. A sterile injectable preparation of the compounds may be in the form of a sterile injectable solution or sterile injectable suspension. Non-toxic, parentally acceptable diluents or solvents such as, for example, 1,3-butanediol can be used to formulate the parenteral compositions. Among the acceptable vehicles and solvents that may be employed are water, Ringer's solution and isotonic sodium chloride solution. In addition, sterile oils also can be employed as a solvent or a suspending medium. For this purpose, any bland fixed oil may be employed, including synthetic monoglycerides or diglycerides. In addition, fatty acids such as oleic acid can be used in the preparation of injectables.

Depending on the vehicle used and the concentration of the drug in the formulation, the parenteral formulation can contain other adjuvants such as local anesthetics, preservatives and buffering agents.

The pharmaceutical compositions according to the disclosure may contain one or more additional therapeutic agents, for example, to increase efficacy and/or to decrease side effects. Examples of such agents include, without limitation, agents to treat or inhibit immunological, inflammatory, autoimmune or allergic disorders.

In an aspect of the disclosure, antibodies are provided that immunoreact with the haptens. The antibodies may be of any of the immunoglobulin subtypes IgA, IgD, IgG or IgM. Antibodies may be produced by convention means and include monoclonal antibodies, polyclonal antibodies, phage display antibodies, and/or human recombinant antibodies. A recombinant antibody can be manipulated or mutated so as to improve its affinity or avidity for the antigen. In an exemplary embodiment, human antibodies or humanized antibodies are used in passive immunization protocols. Methods to humanize murine monoclonal antibodies via several techniques may be used and are well known. Further, methodologies for selecting antibodies with a desired specificity from combinatorial libraries increase the availability of human monoclonal antibodies. Protein engineering may be utilized to prepare human IgG constructs for clinical applications such as passive immunization of a subject. In passive immunization, short-term immunization is achieved by the transfer of antibodies to a subject. The antibodies can be administered in a physiologically acceptable vehicle by any suitable route, e.g., intravenous (IV) or intramuscular (IM). In an exemplary embodiment, active immunization (liposome-hapten conjugate vaccine) and passive immunization (antibodies) may be used in combination in a subject. The effective dose of either the liposome-hapten conjugate vaccine or the antibodies may be the effective dose of either when administered alone. In another exemplary embodiment, the effective dose of either in combination with the other may be less than the amount that would be therapeutically effective if either is administered alone.

### EXAMPLES

### Experimental

All melting points were determined on a Thomas-Hoover melting-point apparatus or a Mettler Toledo MP70 system and are uncorrected. Proton and carbon nuclear magnetic resonance (¹H and ¹³C NMR) spectra were recorded on a Varian Gemini-400 spectrometer in CDCl₃ (unless otherwise noted) with the values given in ppm (TMS as internal standard) and J (Hz) assignments of ¹H resonance coupling. Mass spectra (HRMS) were recorded on a VG 7070E spectrometer or a JEOL SX102a mass spectrometer. Thin layer chromatography (TLC) analyses were carried out on Analtech silica gel GHLF 0.25 mm plates using 10% NH₄OH/MeOH in CHCl₃ or EtOAc in hexanes. Thin layer chromatography (TLC) analyses were also carried out on Analtech silica gel GHLF 0.25 mm plates using various gradients of CHCl3/MeOH containing 1% NH4OH or gradients of n-hexane/EtOAc. Visualization was accomplished under UV light (254 nm) or by staining in an iodine chamber. Flash column chromatography was preformed using a Teledyne Isco CombiFlash Rf+ Lumen with pre-packed silica-gel cartridges. Flash column chromatography was also performed with Fluka silica gel 60 (mesh 220-400) or RediSep Rf normal phase silica gel cartridges.

The optical rotation data were obtained on a PerkinElmer polarimeter model 341. Robertson Microlit Laboratories, Ledgewood, N.J., performed elemental analyses, and the results were within ±0.4% of the theoretical values.

### Example 1. N-((rac-trans)-1-Phenethyl-3-((3-(tritylthio)propanamido)methyl)piperidin-4-yl)-N-phenylpropionamide (14)

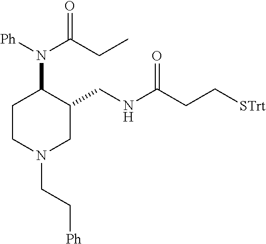

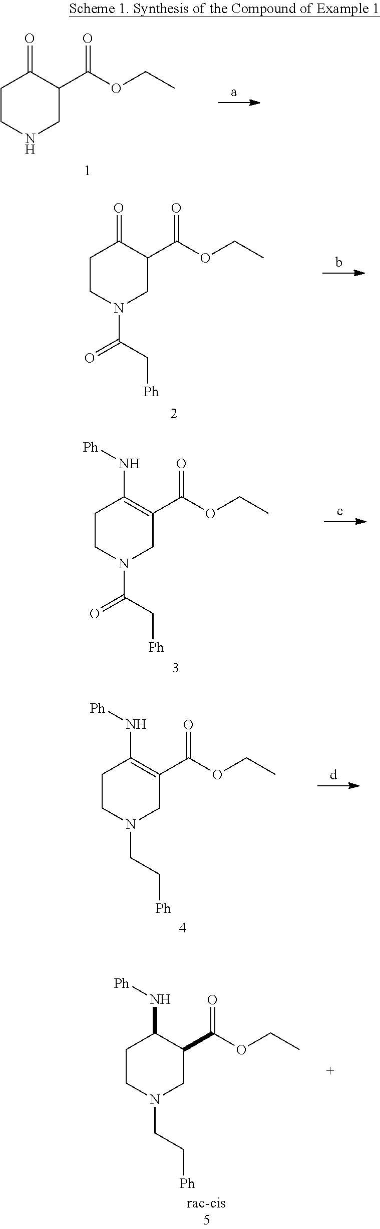 [SMILES: CCOC(=O)C1CNCCC1=O ; CCOC(=O)C1CN(C(=O)Cc2ccccc2)CCC1=O ; CCOC(=O)C1=C(Nc2ccccc2)CCN(C(=O)Cc2ccccc2)C1 ; CCOC(=O)C1=C(Nc2ccccc2)CCN(CCc2ccccc2)C1 ; CCOC(=O)[C@H]1CN(CCc2ccccc2)CC[C@H]1Nc1ccccc1]

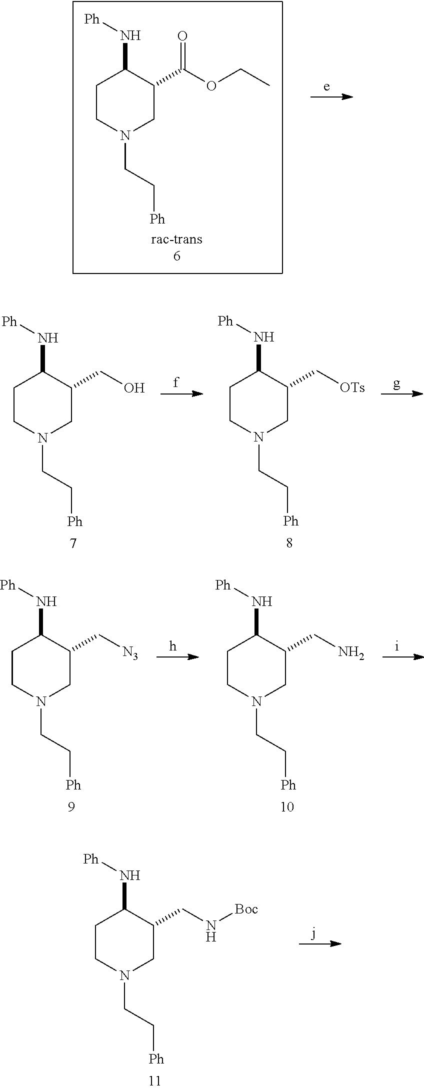 [SMILES: CCOC(=O)[C@@H]1CN(CCc2ccccc2)CC[C@H]1Nc1ccccc1 ; OC[C@@H]1CN(CCc2ccccc2)CC[C@H]1Nc1ccccc1 ; Cc1ccc(S(=O)(=O)OC[C@@H]2CN(CCc3ccccc3)CC[C@H]2Nc2ccccc2)cc1 ; [N-]=[N+]=NC[C@@H]1CN(CCc2ccccc2)CC[C@H]1Nc1ccccc1 ; NC[C@@H]1CN(CCc2ccccc2)CC[C@H]1Nc1ccccc1 ; CC(C)(C)OC(=O)NC[C@@H]1CN(CCc2ccccc2)CC[C@H]1Nc1ccccc1]

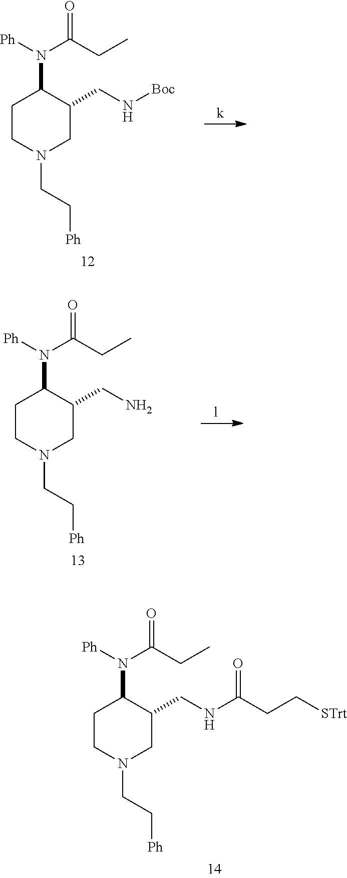

### Example 1-A. Ethyl 4-oxo-1-(2-phenylacetyl)piperidine-3-carboxylate (2)

To a well-stirred suspension of ethyl 4-oxopiperidine-3-carboxylate hydrochloride (1, 24.15 mmol, 5.0 g) and K₂CO₃ (48.30 mmol, 6.68 g) in anhydrous DCM (100 mL), was added phenylacetyl chloride (26.56 mmol, 3.51 mL) slowly over 10 min. After 3 h, the reaction was quenched with 30 mL water. The layers were separated, and the aqueous layer extracted with DCM (3×50 mL). The combined organic extracts were dried over Na₂SO₄, filtered, and concentrated in vacuo. The resultant crude yellow oil was purified by column chromatography on silica gel (50-90% EtOAc in hexanes) to provide 2 as a clear oil (6.31 g, 91% yield). ¹H-NMR (400 MHz, CD₃OD) δ 7.27 (qd, J=14.7, 7.1 Hz, 5H), 4.19 (ddd, J=35.1, 15.6, 8.5 Hz, 4H), 3.82 (d, J=6.1 Hz, 2H), 3.73 (t, J=6.0 Hz, 1H), 3.65 (t, J=5.9 Hz, 1H), 3.30 (s, 1H), 2.35 (t, J=6.0 Hz, 1H), 2.13 (t, J=5.9 Hz, 1H), 1.27 (td, J=12.9, 5.9 Hz, 3H); ¹³C-NMR (101 MHz, CD₃OD) δ 170.96, 170.93, 170.36, 170.07, 169.16, 134.86, 134.63, 128.43, 128.30, 128.16, 126.57, 126.49, 95.26, 60.39, 60.29, 42.25, 41.94, 40.64, 40.12, 38.58, 38.03, 28.41, 27.67, 13.11; HRMS (TOF MS ES+) calcd for C₁₆H₂₀NO₄(M+H⁺) 290.1392, found 290.1394.

### Example 1-B. Ethyl 1-(2-phenylacetyl)-4-(phenylamino)-1,2,5,6-tetrahydropyridine-3-carboxylate (3)

To a solution of 2 (138.3 mmol, 40.0 g) in anhydrous toluene (250 mL) was added a few crystals of p-toluenesulfonic acid and aniline (152.1 mmol, 13.8 mL); the reaction was fitted with a Dean-Stark condenser and heated to reflux. After 16 h, the reaction was cooled to 70° C., and vacuum distilled to remove toluene and excess aniline. The resultant crude oil was redissolved in a 1:1 mixture of hexanes/EtOAc, run through a large plug of silica, and concentrated in vacuo to give 3 as a yellow oil (46 g, 91% yield). ¹H-NMR (400 MHz, CDCl₃) δ 10.57 (d, J=6.3 Hz, 1H), 7.33-7.25 (m, 7H), 7.14 (dd, J=14.0, 7.0 Hz, 1H), 7.01 (d, J=7.9 Hz, 1H), 6.90 (d, J=7.8 Hz, 1H), 4.36 (s, 1H), 4.24 (d, J=5.8 Hz, 1H), 4.18 (qd, J=7.1, 2.7 Hz, 2H), 3.78 (d, J=10.8 Hz, 2H), 3.62 (t, J=5.9 Hz, 1H), 3.42 (t, J=5.7 Hz, 1H), 2.45 (t, J=5.8 Hz, 1H), 2.16 (t, J=5.6 Hz, 1H), 1.30 (td, J=7.1, 2.0 Hz, 3H); ¹³C-NMR (101 MHz, CDCl₃) δ 169.88, 169.37, 168.80, 168.22, 155.71, 153.80, 138.71, 138.47, 134.96, 134.93, 129.12, 129.03, 128.87, 128.79, 128.58, 128.54, 126.79, 126.74, 125.16, 124.87, 124.83, 124.71, 90.91, 89.82, 59.55, 59.48, 43.50, 42.21, 41.59, 41.27, 40.27, 38.32, 27.80, 27.16, 14.52; HRMS (TOF MS ES+) calcd for C₂₂H₂₅N₂O₃ (M+H⁺) 365.1865, found 365.1859.

### Example 1-C. Ethyl 1-phenethyl-4-(phenylamino)-1,2,5,6-tetrahydropyridine carboxylate (4)

To a mechanically stirred, cooled (0° C.) solution of 3 (126.3 mmol, 46.0 g) in anhydrous THF (300 mL) was added lithium aluminum hydride powder (126.3 mmol, 4.8 g) over 1 h. After 3 h, the reaction was diluted with 50 mL diethyl ether, and carefully quenched sequentially with water (4.8 mL), 15% NaOH (4.8 mL) and water (15 mL). After stirring for 30 min, MgSO₄ was added and the mixture further stirred for 15 min, then filtered through a pad of Celite and concentrated in vacuo. The crude material was purified by flash column chromatography on silica gel (20-40% EtOAc in hexanes) to obtain 4 as a clear oil (26 g, 59% yield). ¹H-NMR (400 MHz, CDCl₃) δ 10.62 (s, 1H), 7.32-7.07 (m, 10H), 4.20 (q, J=7.1 Hz, 2H), 3.36 (s, 2H), 2.88 (dd, J=10.1, 6.0 Hz, 2H), 2.73 (dd, J=10.2, 6.0 Hz, 2H), 2.59 (t, J=5.4 Hz, 2H), 2.54 (d, J=5.1 Hz, 2H), 1.31 (t, J=7.1 Hz, 3H); ¹³C-NMR (101 MHz, CDCl₃) δ 169.13, 154.65, 140.27, 139.30, 128.97, 128.69, 128.39, 126.04, 124.90, 124.64, 91.50, 60.12, 59.22, 51.56, 49.56, 34.08, 28.63, 14.62; HRMS (TOF MS ES+) calcd for C₂₂H₂₇N₂O₂ (M+H⁺) 351.2073, found 351.2071.

### Example 1-D. Ethyl-(rac)-cis-1-phenethyl-4-(phenylamino)piperidine-3-carboxylate (5) and Ethyl-(rac)-trans-1-phenethyl-4-(phenylamino)piperidine-3-carboxylate (6)

To a cooled (0° C.) suspension of NaBH₄ (251.4 mmol, 9.51 g) in anhydrous acetonitrile (300 mL) was added glacial acetic acid (817.2 mmol, 49 mL) in anhydrous acetonitrile (100 mL) slowly over 30 min. After stirring for an additional 30 min, a solution of 4 (62.9 mmol, 22 g) in anhydrous acetonitrile (100 mL) was added slowly over 30 min, while maintaining a reaction temperature of 0° C. The reaction was allowed to warm to room temperature for 16 h, upon which time the reaction was quenched with water (15 mL), and concentrated in vacuo. The aqueous suspension was adjusted to pH 10 with NH₄OH and extracted with CHCl₃ (3×200 mL), the combined organic extracts dried over Na₂SO₄, filtered, and concd in vacuo. The crude material was purified by flash column chromatography on silica gel (20-40% EtOAc in hexanes) to afford 5 (rac-cis) as a white amorphous solid (13.0 g, 49% yield). mp 92-94° C. and 6 (rac-trans) as a clear oil (1.3 g, 6% yield). Compound 5 (rac-cis): ¹H-NMR (400 MHz, CDCl₃) δ 7.30 (dd, J=14.8, 7.1 Hz, 2H), 7.26-7.15 (m, 5H), 6.71-6.67 (m, 1H), 6.68-6.63 (m, 2H), 4.17-4.06 (m, 2H), 3.79-3.73 (m, 1H), 3.21-3.06 (m, 1H), 2.95 (dd, J=6.3, 3.2 Hz, 1H), 2.83-2.77 (m, 2H), 2.75-2.51 (m, 4H), 2.45-2.37 (m, 1H), 2.07 (ddt, J=13.0, 8.7, 4.3 Hz, 1H), 1.89-1.83 (m, 1H), 1.22 (dt, J=15.5, 8.2 Hz, 3H); ¹³C-NMR (101 MHz, CDCl₃) δ 172.70, 146.91, 140.34, 129.30, 128.68, 128.33, 126.00, 117.56, 113.74, 60.41, 60.15, 53.64, 51.86, 50.34, 44.20, 33.63, 28.85, 14.13; HRMS (TOF MS ES+) calcd for C₂₂H₂₉N₂O₂(M+H⁺) 353.2229, found 353.2233. Compound 6 (rac-trans): ¹H-NMR (400 MHz; CDCl₃) δ 7.27 (q, J=7.3 Hz, 2H), 7.20-7.18 (m, 3H), 7.14 (t, J=7.9 Hz, 2H), 6.68 (t, J=7.3 Hz, 1H), 6.62 (d, J=7.8 Hz, 2H), 4.09-4.00 (m, 2H), 3.64 (td, J=10.2, 4.1 Hz, 1H), 3.08 (dd, J=11.1, 1.1 Hz, 1H), 2.94 (dd, J=11.4, 0.8 Hz, 1H), 2.86-2.77 (m, 3H), 2.66-2.57 (m, 3H), 2.44 (t, J=10.8 Hz, 1H), 2.27-2.15 (m, 2H), 1.44 (qd, J=12.0, 3.5 Hz, 1H), 1.16 (t, J=7.1 Hz, 3H); ¹³C-NMR (101 MHz, CDCl₃) δ 173.02, 146.89, 140.17, 129.21, 128.65, 128.36, 126.04, 117.68, 113.62, 60.72, 59.91, 54.73, 52.61, 52.22, 49.42, 43.54, 33.69, 31.89, 14.08; HRMS (TOF MS ES+) calcd for C₂₂H₂₉N₂O₂(M+H⁺) 353.2229, found 353.2230.

### Example 1-E. rac-trans-1-Phenethyl-4-(phenylamino)piperidin-3-yl)methanol (7)

To a cooled (0° C.) solution of 6 (rac-trans) (8.5 mmol, 3.0 g) in anhydrous THF (50 mL) was added a 1.0M solution of lithium aluminum hydride in THF (1.1 equiv 9.4 mmol, 9.4 mL) slowly over 10 min. After 2 h, the reaction was diluted with diethyl ether and quenched sequentially with water (3504), 15% NaOH (350 μL) and water (1.0 mL). After stirring for 15 min, MgSO₄ was added and the mixture further stirred for 15 min, then filtered through a pad of Celite and concentrated in vacuo to give a crude clear oil. Purification by flash column chromatography on silica gel (10% NH₄OH/MeOH in CHCl₃, gradient 0-10%) afforded 7 as a clear oil (2.2 g, 84% yield). ¹H-NMR (400 MHz; CDCl₃) δ 7.29-7.24 (m, 2H), 7.20-7.15 (m, 5H), 6.74 (t, J=7.3 Hz, 1H), 6.66 (d, J=7.8 Hz, 2H), 3.79 (dd, J=10.9, 6.0 Hz, 1H), 3.69 (dd, J=10.9, 4.3 Hz, 1H), 3.41 (s, 1H), 3.28 (d, J=3.9 Hz, 1H), 3.13 (s, 1H), 2.99 (dt, J=11.4, 1.7 Hz, 1H), 2.87 (dd, J=10.4, 4.6 Hz, 1H), 2.80 (dd, J=9.8, 6.3 Hz, 2H), 2.62-2.58 (m, 2H), 2.17 (td, J=8.9, 2.7 Hz, 2H), 2.06 (t, J=10.6 Hz, 1H), 1.84 (dddd, J=13.8, 9.2, 5.2, 4.3 Hz, 1H), 1.50-1.41 (m, 1H); ¹³C-NMR (101 MHz, CDCl₃) δ 146.90, 140.18, 129.36, 128.62, 128.37, 126.04, 118.46, 114.45, 65.75, 60.40, 55.51, 54.61, 52.19, 43.56, 33.74, 32.09; HRMS (TOF MS ES+) calcd for C₂₀H₂₈N₂O (M+H⁺) 311.2123, found 311.2122.

### Example 1-F. (rac-trans) 1-Phenethyl-4-(phenylamino)piperidin-3-yl)methyl 4-methylbenzenesulfonate (8)

To a solution of 7 (3.86 mmol, 1.2 g), tosyl chloride (4.25 mmol, 811 mg) and DMAP (catalytic, ca 10 mg) in anhydrous DCM (30 mL) was added triethylamine (5.79 mmol, 0.81 mL), and the reaction stirred at rt overnight. After 18 h, the reaction was quenched with saturated aq. NH₄Cl, the layers separated and the aqueous layer extracted with DCM (3×25 mL), the combined organic extracts dried over Na₂SO₄ and concentrated under vacuum to give crude product. Purification via flash column chromatography on silica gel (0-100% EtOAc in hexanes) afforded 8 as a white foam (1.75 g, 99% yield). ¹H NMR (CDCl₃, 400 MHz): δ 7.69 (d, J=8.0 Hz, 2H), 7.32-7.13 (m, 9H), 6.71 (t, J=7.2 Hz, 1H), 6.51 (d, J=8.0 Hz, 2H), 4.24 (dd, J=9.6, 3.2 Hz, 1H), 4.03 (d, J=9.6, 6.8 Hz, 1H), 3.18 (br, 2H), 3.09 (brd, J=11.2 Hz, 1H), 2.93 (brd, J=11.2 Hz, 1H), 2.79-2.75 (brm, 2H), 2.62-2.58 (brm, 2H), 2.38 (s, 3H), 2.14-2.05 (m, 3H), 1.91 (br, 1H), 1.41-1.33 (brm, 1H); ¹³C NMR (CDCl₃, 100 MHz): [symbol] 147.0, 144.8, 140.3, 132.6, 129.9, 129.4, 128.8, 128.5, 128.0, 126.2, 117.8, 113.5, 70.5, 60.4, 55.5, 52.6, 50.8, 42.5, 33.9, 32.2, 21.7; HRMS (TOF MS ES+) calcd for C₂₇H₃₃N₂O₃S (M+H⁺): 465.2206; found: 465.2207.

### Example 1-G. (rac-trans) 3-(Azidomethyl)-1-phenethyl-N-phenylpiperidin-4-amine (9)

To a solution of 8 (3.86 mmol, 1.74 g) in anhydrous DMF (10 mL) was added sodium azide (15.44 mmol, 1.00 g) and heated to 60° C. After 18 h, the reaction was cooled to rt, quenched with H₂O and extracted with toluene (3×25 mL), the combined toluene extracts washed with H₂O (5×20 mL), then brine, dried over Na₂SO₄ and concentrated under vacuum to give the product 9 as a yellow oil (1.3 g, quantitative yield). ¹H NMR (CDCl₃, 400 MHz): δ 7.35-7.31 (m, 2H), 7.26-7.19 (m, 5H), 6.74 (t, J=7.2 Hz, 1H), 6.60 (d, J=8.0 Hz, 2H), 3.65 (dd, J=12.4, 3.6 Hz, 1H), 3.41 (dd, J=12.4, 7.6 Hz, 1H), 3.43-3.30 (br, 1H), 3.21 (brm, 1H), 3.15 (brd, J=11.6 Hz, 1H), 4.50 (brd, J=11.2 Hz, 1H), 2.87-2.83 (m, 2H), 2.68-2.64 (m, 2H), 2.20-2.14 (m, 2H), 2.08 (t, J=11.2 Hz, 1H), 1.87-1.80 (m, 1H), 1.51-1.41 (m, 1H); ¹³C NMR (CDCl₃, 100 MHz): [symbol] 147.3, 140.3, 129.5, 128.8, 128.5, 126.2, 117.7, 113.4, 60.5, 56.5, 52.7, 52.6, 52.1, 43.1, 33.9, 32.4; HRMS (TOF MS ES+) calcd for C₂₀H₂₆N₅ (M+H⁺): 336.2183;

### Example 1-H. (rac-trans) 3-(Aminomethyl)-1-phenethyl-N-phenylpiperidin-4-amine (10)

To a solution of 9 (3.86 mmol, 1.3 g) in THF (30 mL) and H₂O (1.5 mL) was added PPh₃ (4.63 mmol, 1.2 g) and stirred at 60° C. overnight. After 18 h, the reaction was concentrated under vacuum and 10 was used without any further purification owing to the coelution of the product with triphenylphosphine oxide.

### Example 1-I. tert-Butyl (((rac-trans) 1-phenethyl-4-(phenylamino)piperidin-3-yl)methyl)carbamate (11)

To a solution of 10 (2.26 mmol, 700 mg) in anhydrous DCM (20 mL) was added Boc₂O (2.49 mmol, 0.57 mL), DMAP (catalytic, ca 10 mg) and triethylamine (4.52 mmol, 0.63 mL) and stirred at rt overnight. After 18 h, the reaction was quenched with H₂O and extracted with DCM (3×25 mL), dried over Na₂SO₄ and concentrated under vacuum. Purification by flash column chromatography on silica gel (10% NH₄OH/MeOH in CHCl₃, gradient 0-10%) afforded 11 as a yellow oil (450 mg, 28%). ¹H NMR (CDCl₃, 400 MHz): δ 7.31-7.26 (m, 3H), 7.21-7.15 (m 4H), 6.70 (t, J=7.2 Hz, 1H), 6.62 (d, J=8.0 Hz, 2H), 4.85 (br, 1H), 3.53-3.38 (brm, 2H), 3.10-3.02 (brm, 3H), 2.95 (brd, J=10.4 Hz, 1H), 2.83-2.79 (brm, 2H), 2.63-2.58 (brm, 2H), 2.09-2.08 (m, 2H), 1.95 (brt, J=10.8 Hz, 1H), 1.77 (br, 1H), 1.43 (brs, 9h), 1.43-1.37 (br, 1H). HRMS (TOF MS ES+) calcd for C₂₅H₃₆N₃O₂(M+H⁺): 410.2802; found: 410.2802.

### Example 1-J. tert-Butyl (((rac-trans) 1-Phenethyl-4-(N-phenylpropionamido)piperidin-3-yl)methyl)carbamate (12)

To a solution of 11 (1.1 mmol, 450 mg) in 20 mL anhydrous toluene was added triethylamine (3.3 mmol, 0.46 mL) and propionyl chloride (1.65 mmol, 0.15 mL), and the reaction stirred at 40° C. After 24 h, the reaction was quenched with H₂O, extracted with EtOAc (3×25 mL), dried over Na₂SO₄ and concentrated under vacuum. Purification via flash column chromatography on silica gel (0-100% EtOAc in hexanes) afforded 12 as a yellow oil (280 mg, 55%). ¹H NMR (CDCl₃, 400 MHz): δ 7.42-7.34 (m, 3H), 7.24 (t, J=7.2 Hz, 2H), 7.18-7.12 (m, 4H), 7.07 (brd, J=6.8 Hz, 1H), 5.67 (brm, 1H), 4.62 (brtd, J=12.0, 6.8 Hz, 1H), 3.66-3.60 (brm, 1H), 3.11-3.08 (m, 1H), 3.01-2.96 (m, 2H), 2.78-2.66 (m, 2H), 2.60-2.48 (m, 2H), 2.15-1.91 (m, 4H), 1.80-1.66 (m, 2H), 1.46 (brs, 9H), 1.40-1.26 (m, 1H), 1.02 (t, J=7.6 Hz, 3H); ¹³C NMR (CDCl₃, 100 MHz): δ 175.1, 156.5, 140.1, 138.4, 131.0, 129.9, 129.6, 129.4, 128.73, 128.66, 128.5, 126.2, 79.0, 60.4, 56.1, 52.8, 52.7, 40.2, 39.9, 33.5, 30.5, 28.64, 28.60, 9.95; HRMS (TOF MS ES+) calcd for C₂₈H₄₀N₃O₃(M+H⁺): 466.3064; found: 466.3066.

### Example 1-K. N-((rac-trans) 3-(Aminomethyl)-1-phenethylpiperidin-4-yl)-N-phenylpropionamide (13)

To a cooled (0° C.) solution of 12 (0.56 mmol, 260 mg) in anhydrous CHCl₃ (10 mL) was added trifluoroacetic acid (5.6 mmol, 0.43 mL) dropwise, then stirred at 0° C. for 15 min and allowed to warm to rt overnight. After 16 h, the reaction was quenched with saturated aq. NaHCO₃ and extracted with CHCl₃ (3×25 mL), dried over Na₂SO₄ and concentrated under vacuum. Purification via flash column chromatography on silica gel (10% NH₄OH/MeOH in CHCl₃, gradient 0-10%) afforded 13 as a yellow oil (178 mg, 87%). ¹H-NMR (400 MHz; CDCl₃): δ 7.40-7.34 (m, 3H), 7.24 (q, J=4.6 Hz, 2H), 7.14 (dd, J=13.6, 7.1 Hz, 3H), 7.06 (d, J=7.2 Hz, 2H), 4.70 (t, J=10.1 Hz, 1H), 3.10-3.07 (m, 1H), 2.94 (d, J=11.4 Hz, 1H), 2.85 (t, J=5.0 Hz, 2H), 2.76-2.68 (m, 2H), 2.53 (t, J=8.3 Hz, 2H), 2.13-2.06 (m, 2H), 1.98-1.89 (m, 2H), 1.72 (dt, J=9.2, 3.3 Hz, 1H), 1.62 (s, 1H), 1.40 (qd, J=12.3, 3.7 Hz, 1H), 1.00 (t, J=7.4 Hz, 3H). ¹³C-NMR (101 MHz; CDCl₃): δ 174.39, 140.13, 138.48, 131.06, 129.58, 129.45, 129.26, 128.60, 128.36, 126.00, 60.57, 56.72, 53.16, 42.54, 41.32, 33.80, 30.56, 28.49, 9.75. HRMS (TOF MS ES+) calcd for C₂₃H₃₂N₃O (M+H⁺): 366.2540; found: 366.2540.

### Example 1-L. N-((rac-trans) 1-Phenethyl-3-((3-(tritylthio)propanamido)methyl)piperidin-4-yl)-N-phenylpropionamide (14)

To a cooled (0° C.) solution of 13 (0.27 mmol, 100 mg) in anhydrous DCM (15 mL) was added, sequentially, TBTU (0.82 mmol, 264 mg), 3-(tritylthio)propionic acid (0.82 mmol, 286 mg) and triethylamine (1.09 mmol, 0.15 mL) and stirred overnight allowing the reaction to warm to rt. After 18 h, the reaction was quenched with H₂O and extracted with DCM (3×25 mL), dried over Na₂SO₄ and concentrated under vacuum. Purification via flash column chromatography on silica gel (10% NH₄OH/MeOH in CHCl3, gradient 1-10%) afforded 14 as a white foam (78 mg, 41%). ¹H-NMR (400 MHz; CDCl₃): δ 7.39 (t, J=8.9 Hz, 8H), 7.25-7.13 (m, 11H), 7.05 (d, J=7.3 Hz, 3H), 6.94 (d, J=8.0 Hz, 1H), 4.55 (t, J=10.4 Hz, 1H), 3.93 (t, J=11.7 Hz, 1H), 3.01 (d, J=11.3 Hz, 1H), 2.86-2.82 (m, 2H), 2.78 (s, 1H), 2.67-2.57 (m, 3H), 2.43 (ddt, J=16.4, 11.0, 5.5 Hz, 3H), 2.16 (dtd, J=21.5, 14.4, 7.1 Hz, 2H), 2.05-1.89 (m, 4H), 1.73-1.65 (m, 2H), 1.29-1.23 (m, 2H), 1.00 (t, J=7.4 Hz, 3H). ¹³C-NMR (101 MHz; CDCl₃): δ 175.26, 171.17, 144.74, 140.03, 138.12, 130.78, 129.62, 129.31, 129.23, 128.60, 128.26, 127.84, 126.56, 125.90, 66.67, 60.19, 55.85, 52.81, 39.83, 38.58, 38.37, 35.83, 33.62, 30.69, 28.52, 28.04, 9.81. HRMS (TOF MS ES+) calcd for C₄₅H₅₀N₃O₂ (M+H⁺): 696.3624; found 696.3616. Calcd for C₄₅H₄₉N₃O₂S·0.1 CHCl₃: C, 76.52; H, 6.99; N, 5.94; found: C, 76.22; H, 7.14; N, 6.21.

### Example 2. N-phenyl-N-(1-(4-(3-(tritylthio)propanamido)phenethyl)piperidin-4-yl)propionamide (19)

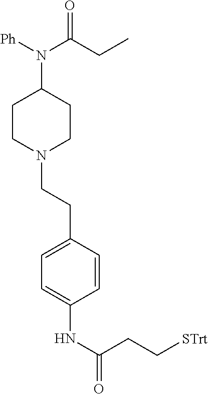

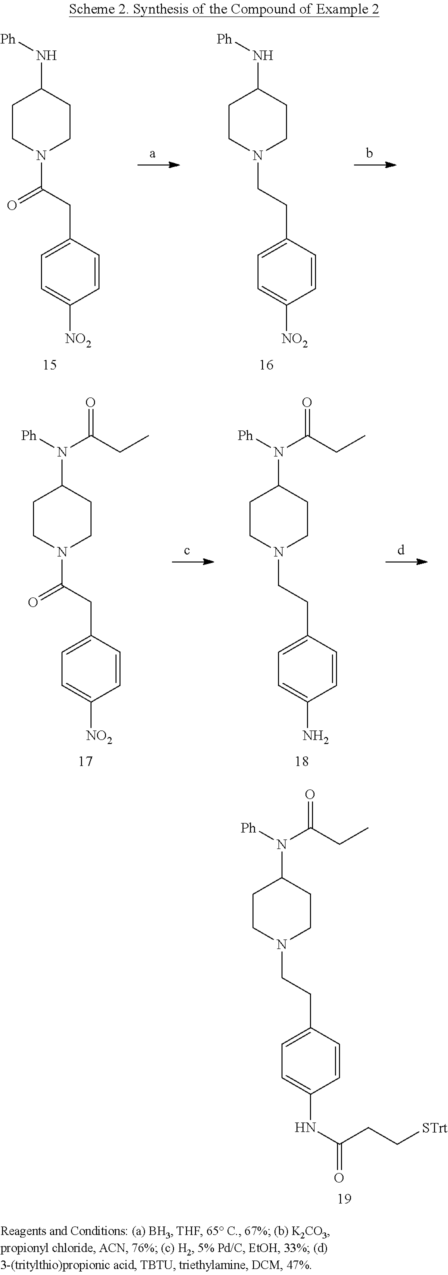

### Example 2-A. 2-(4-Nitrophenyl)-1-(4-(phenylamino)piperidin-1-yl)ethan-1-one (15)

Prepared as previously reported in Burke, T. R. et al., Journal of Medicinal Chemistry (1984) 27(12):1570-1574.

### Example 2-B. 1-(4-Nitrophenethyl)-N-phenylpiperidin-4-amine (16)

To a solution of 15 (50.0 mmol, 17.0 g) in anhydrous THF (200 mL) was added a 1M solution of BH₃ in THF (150 mmol, 150 mL), and the reaction heated to reflux. After 1.5 h, the reaction was slowly quenched with MeOH and concentrated under vacuum. The resultant residue was suspended in 1N HCl and refluxed for 3 h, then cooled to 0° C. and basified to approximately pH 9.0 with 28% NH₄OH, then extracted with CHCl₃ (3×100 mL), dried over Na₂SO₄ and concentrated under vacuum. The crude oil was dissolved in CHCl₃ and distilled, replacing the volume with isopropanol. Once the distillate temp reached 80° C., the solution was cooled and stirred for 2 h, then filtered to collect the product 16 as orange crystals (10.9 g, 67%). mp: 92-94° C. ¹H-NMR (400 MHz; CDCl₃): δ 8.17 (d, J=8.4 Hz, 2H), 7.41 (d, J=8.3 Hz, 2H), 7.15 (t, J=7.7 Hz, 2H), 6.69 (t, J=7.3 Hz, 1H), 6.56 (d, J=8.0 Hz, 2H), 4.46 (d, J=13.7 Hz, 1H), 3.82 (d, J=7.4 Hz, 3H), 3.51-3.46 (m, 2H), 3.19 (t, J=12.5 Hz, 1H), 2.91 (t, J=12.4 Hz, 1H), 2.05 (t, J=12.7 Hz, 2H), 1.37-1.28 (m, 1H), 1.23-1.14 (m, 1H). ¹³C-NMR (101 MHz; CDCl₃): δ 167.77, 146.94, 146.39, 142.68, 129.83, 129.38, 123.81, 117.77, 113.26, 49.73, 44.83, 40.91, 40.41, 32.73, 32.05.

### Example 2-C. N-(1-(4-Nitrophenethyl)piperidin-4-yl)-N-phenylpropionamide (17)

To a solution of 16 (3.07 mmol, 1.0 g) in anhydrous ACN (30 mL) was added K₂CO₃ (6.15 mmol, 0.85 g) following by propionyl chloride (3.38 mmol, 0.3 mL). After 2 h, the reaction was quenched with H₂O and extracted with CHCl3 (3×25 mL), dried over Na₂SO₄ and concentrated under vacuum. The crude residue was dissolved in hot cyclohexane and allowed to slowly cool to rt, and stirred for 1 h, then filtered to collect the product 17 as white crystals (0.89 g, 76% yield). mp: 120-122° C. ¹H-NMR (400 MHz; CDCl₃): δ 8.08 (d, J=8.4 Hz, 2H), 7.39-7.34 (m, 3H), 7.27 (d, J=8.4 Hz, 2H), 7.05 (d, J=6.9 Hz, 2H), 4.65 (t, J=12.2 Hz, 1H), 2.93 (d, J=11.3 Hz, 2H), 2.79 (t, J=7.9 Hz, 2H), 2.53 (t, J=8.0 Hz, 2H), 2.15 (t, J=11.6 Hz, 2H), 1.90 (q, J=7.4 Hz, 2H), 1.78 (d, J=11.8 Hz, 2H), 1.41-1.33 (m, 2H), 0.98 (t, J=7.4 Hz, 3H). ¹³C-NMR (101 MHz; CDCl₃): δ 173.51, 148.21, 146.43, 138.77, 130.34, 129.39, 129.27, 128.27, 123.57, 59.43, 53.04, 52.03, 33.64, 30.51, 28.48, 9.56.

### Example 2-D. N-(1-(4-Aminophenethyl)piperidin-4-yl)-N-phenylpropionamide (18)

A solution of 17 (0.66 mmol, 250 mg) in EtOH (15 mL) was transferred to a pressure bottle, Escat 103 (5% Pd/C, 0.05 g) was added and the bottle pressurized to 50 psi H2 in a Parr shaker. After 2 h, the reaction was filtered through celite and concentrated under vacuum. Analytically pure product was obtained by crystallization from toluene to give 18 as clear needles (84 mg, 33%). ¹H-NMR (400 MHz; CDCl₃): δ 7.35 (q, J=7.4 Hz, 3H), 7.05 (d, J=7.0 Hz, 2H), 6.91 (d, J=8.0 Hz, 2H), 6.57 (d, J=8.0 Hz, 2H), 4.69-4.62 (m, 1H), 4.64 (t, J=0.7 Hz,), 3.52 (s, 2H), 2.96 (d, J=11.4 Hz, 2H), 2.59 (dd, J=10.4, 6.0 Hz, 2H), 2.44 (dd, J=10.9, 5.5 Hz, 2H), 2.11 (t, J=11.7 Hz, 2H), 1.90 (q, J=7.4 Hz, 2H), 1.77 (d, J=11.9 Hz, 2H), 1.44-1.35 (m, 2H), 0.99 (t, J=7.4 Hz, 3H). ¹³C-NMR (101 MHz; CDCl₃): δ 173.47, 144.39, 138.81, 130.40, 130.15, 129.35, 129.22, 128.19, 115.20, 60.85, 53.08, 52.13, 32.92, 30.54, 28.49, 9.59.

### Example 2-E. N-Phenyl-N-(1-(4-(3-(tritylthio)propanamido)phenethyl)piperidin-4-yl)propionamide (19)

To a solution of 18 (140 mg, 0.4 mmol) in anhydrous DCM (10 mL) was added TBTU (1.2 mmol, 385 mg), 3-(tritylthio)propionic acid (1.2 mmol, 418 mg) and triethylamine (1.6 mmol, 0.22 mL). After 24 h, the reaction was quenched with H₂O and extracted with DCM (3×10 mL), dried over Na₂SO₄ and concentrated under vacuum. Purification via flash column chromatography on silica gel (isocratic, 50:49:1 DCM:ACN:28% NH₄OH) gave the product 19 as a white foam (132 mg, 47%). ¹H-NMR (400 MHz; CDCl₃): δ 7.42-7.30 (m, 10H), 7.26 (t, J=7.8 Hz, 6H), 7.19 (t, J=7.1 Hz, 3H), 7.10-7.03 (m, 5H), 4.64 (t, J=12.1 Hz, 1H), 2.93 (d, J=11.0 Hz, 2H), 2.64 (t, J=8.0 Hz, 2H), 2.55 (t, J=7.2 Hz, 2H), 2.45 (dd, J=10.3, 5.8 Hz, 2H), 2.14-2.06 (m, 4H), 1.90 (q, J=7.4 Hz, 2H), 1.76 (d, J=12.0 Hz, 2H), 1.67 (s, 1H), 1.38 (q, J=11.0 Hz, 2H), 0.99 (t, J=7.4 Hz, 3H). ¹³C-NMR (101 MHz; CDCl₃): δ 173.55, 173.55, 169.05, 169.05, 144.56, 144.56, 138.73, 138.73, 136.19, 136.19, 135.71, 135.71, 130.37, 130.37, 129.54, 129.54, 129.25, 129.25, 129.01, 129.01, 128.24, 128.24, 127.94, 127.94, 126.70, 126.70, 119.88, 119.88, 60.42, 60.42, 53.03, 53.03, 52.10, 52.10, 36.69, 36.69, 33.17, 33.17, 30.52, 30.52, 28.51, 28.51, 27.65, 27.65, 9.61, 9.61. HRMS (TOF MS ES+) calcd for C₄₄H₄₇N₃O₂S (M+H⁺): 682.3467; found 682.3475. Calcd for C₄₄H₄₇N₃O₂S·0.47 CHCl₃: C, 71.38; H, 6.39; N, 5.60; found: C, 71.37; H, 6.46; N, 5.62.

### Example 3. N-Phenyl-N-(1-(4-((3-(tritylthio)propanamido)methyl)phenethyl)piperidin-4-yl)propionamide (34, trityl capped para-AmMeFenHap)

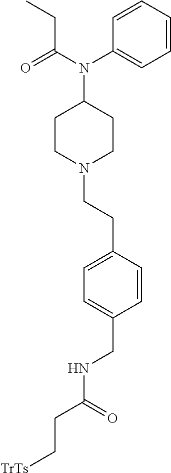

Compound 34 was synthesized according to Scheme 3 below.

### Example 4. N-Phenyl-N-(1-(4-(2-(3-(tritylthio)propanamido)ethyl)phenethyl)piperidin-4-yl)propionamide (35, trityl capped para-AmEtFenHap)

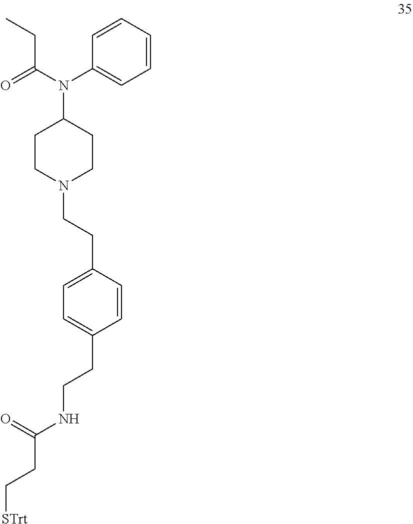

Compound 35 was synthesized according to Scheme 3 below.

### Example 5. N-Phenyl-N-(1-(4-(3-(3-(tritylthio)propanamido)propyl)phenethyl)piperidin-4-yl)propionamide (36, trityl capped para-AmPrFenHap)

Compound 36 was synthesized according to Scheme 3 below.

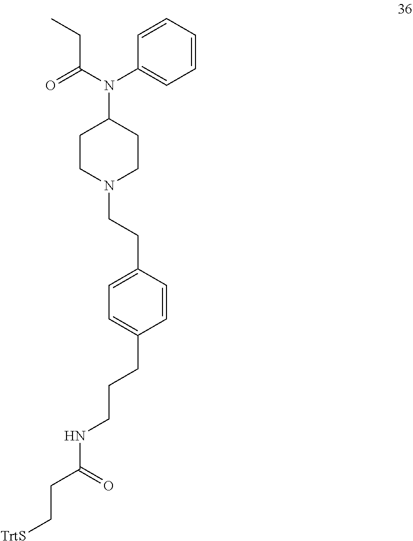

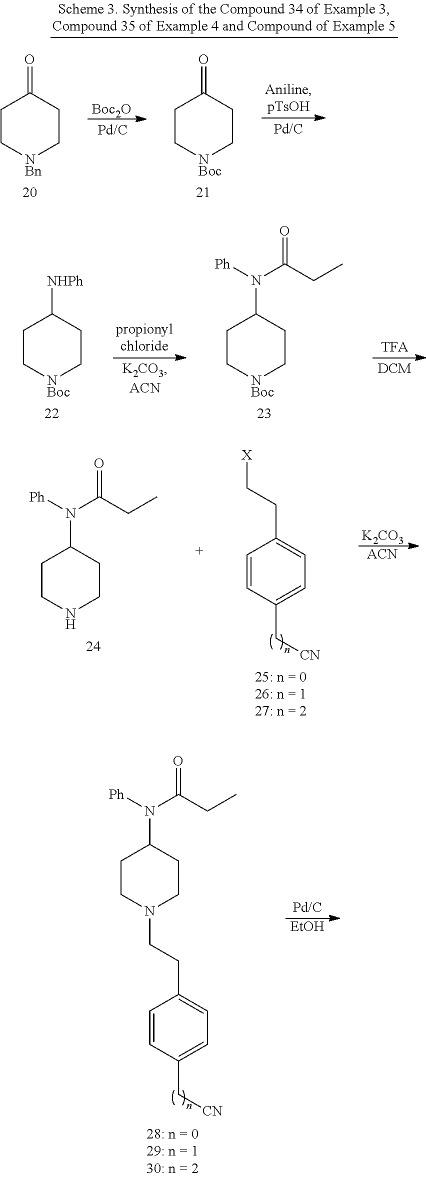

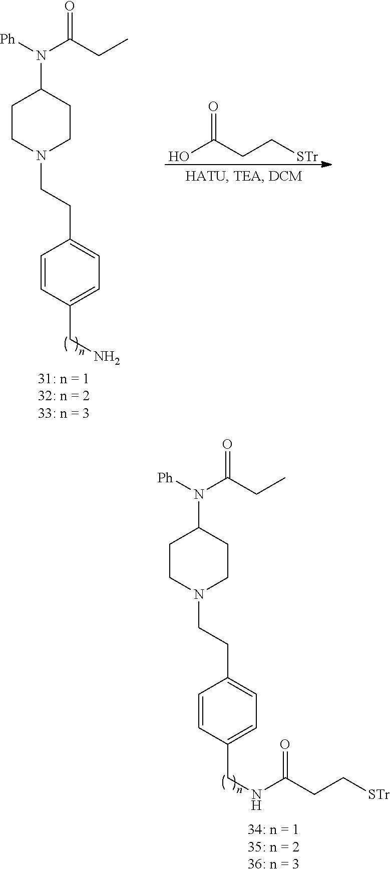

### Example 3-A. tert-Butyl 4-Oxopiperidine-1-carboxylate (21)

N-Benzyl-4-piperidone (20, 18.9 g, 100 mmol) was dissolved in EtOAc (100 mL) and transferred to a Parr shaker vessel, charged with Boc₂O (27.6 mL, 120 mmol) and Escat 103 (5% Pd/C, 1.0 g) and shaken for 24 h under 50 psi H2. The reaction mixture was filtered through celite and concentrated under vacuum. The resultant residue was hexane (100 mL) and concentrated until approximately half of the solvent was removed resulting in the formation of white crystals. This process was repeated 3 times, and the product collected via filtration and washed with hexane to afford tert-butyl 4-oxopiperidine-1-carboxylate (21) as white crystals (16.0 g, 80%). ¹H NMR (400 MHz; CDCl₃) δ 3.68 (t, J=6.2 Hz, 4H), 2.40 (t, J=6.2 Hz, 4H), 1.46 (s, 9H). ¹³C-NMR (101 MHz, CDCl₃): δ 207.79, 154.42, 80.40, 41.13, 28.32. mp: 74-76° C.

### Example 3-B. tert-Butyl 4-(Phenylamino)piperidine-1-carboxylate (22)

A solution of tert-butyl 4-oxopiperidine-1-carboxylate (21, 16.0 g, 80.4 mmol) in EtOAc (40 mL) was transferred to a Parr shaker vessel and charged with aniline (7.1 mL, 80.4 mmol) pTsOH (cat., 100 mg) and Escat 103 (5% Pd/C, 500 mg), and shaken for 24 h under 50 psi H2. The reaction mixture was filtered through celite and concentrated under vacuum. The product was crystallized from cyclohexane resulting in tert-butyl 4-(phenylamino)piperidine-1-carboxylate (22) as a white crystalline powder (14.6 g, 66%). ¹H NMR (400 MHz; CDCl₃) δ 7.15 (t, J=7.9 Hz, 2H), 6.68 (t, J=7.3 Hz, 1H), 6.59-6.57 (m, 2H), 4.02 (s, 2H), 3.49-3.37 (m, 2H), 2.91 (t, J=11.9 Hz, 2H), 2.04-2.00 (m, 2H), 1.45 (s, 9H), 1.36-1.26 (m, 2H). ¹³C-NMR (101 MHz, CDCl₃): δ 154.76, 146.72, 129.33, 117.45, 113.26, 79.57, 50.07, 32.37, 28.41. HRMS-ESI (m/z): [M+H]⁺ calcd. for C₁₆H₂₅N₂O₂ 277.1916, found 277.1916. mp: 134-136° C.

### Example 3-C. tert-Butyl 4-(N-Phenylpropionamido)piperidine-1-carboxylate (23)

To solution of tert-butyl 4-(phenylamino)piperidine-1-carboxylate (22, 4.1 g, 14.9 mmol) in acetonitrile (50 mL) was added K₂CO₃ (6.2 g, 44.6 mmol) and propionyl chloride (1.4 mL, 16.3 mmol) and heated to 50° C. for 16 h. The reaction was then cooled, diluted with H₂O (50 mL) and extracted with CHCl₃ (3×50 mL), dried with Na₂SO₄ and concentrated under vacuum. The resultant residue was purified via flash chromatography (EtOAc in hexane, gradient 0-40%) to afford tert-butyl 4-(N-phenylpropionamido)piperidine-1-carboxylate (23) as a white solid (3.7 g, 77%). ¹H NMR (400 MHz; CDCl₃) δ 7.38 (d, J=7.0 Hz, 3H), 7.04 (dd, J=7.5, 1.8 Hz, 2H), 4.75 (tt, J=12.2, 3.7 Hz, 1H), 4.08 (s, 2H), 2.76 (t, J=12.3 Hz, 2H), 1.90 (q, J=7.4 Hz, 2H), 1.74 (d, J=11.9 Hz, 2H), 1.67 (s, 1H), 1.36 (s, 9H), 1.24-1.14 (m, 2H), 0.99 (t, J=7.4 Hz, 3H). ¹³C-NMR (101 MHz, CDCl₃): δ 173.48, 154.52, 138.69, 130.24, 129.36, 128.40, 79.48, 52.14, 30.47, 28.45, 28.32, 9.55. HRMS-ESI (m/z): [M+H]⁺ calcd. for C₁₉H₂₈N₂O₃ 355.1998, found 355.2000. mp 134-136° C.

### Example 3-D. N-Phenyl-N-(piperidin-4-yl)propionamide (24)

To a solution of tert-butyl 4-(N-phenylpropionamido)piperidine-1-carboxylate (23, 3.7 g, 11.1 mmol) in anhydrous DCM (30 mL) was added trifluoroacetic acid (4.0 mL, 52 mmol) and stirred for 16 h. The reaction was quenched with saturated aq. NaHCO₃, extracted with CHCl₃ (3×25 mL), dried over Na₂SO₄ and concentrated under vacuum. The resulting residue was purified via flash chromatography (10% NH₄OH/MeOH in CHCl₃, gradient 0-10%) to afford N-phenyl-N-(piperidin-4-yl)propionamide (24) as a clear oil (2.5 g, 96%). ¹H NMR (400 MHz; CDCl₃) δ 7.38-7.32 (m, 3H), 7.05-7.02 (m, 2H), 4.70 (tt, J=12.1, 3.9 Hz, 1H), 3.01 (dd, J=10.0, 2.1 Hz, 2H), 2.68 (td, J=12.3, 2.3 Hz, 2H), 1.88 (q, J=7.4 Hz, 2H), 1.77-1.73 (m, 3H), 1.22 (qd, J=12.3, 4.1 Hz, 2H), 0.97 (t, J=7.5 Hz, 3H). ¹³C-NMR (101 MHz, CDCl₃): δ 173.31, 138.89, 130.33, 129.21, 128.21, 52.28, 46.05, 31.82, 28.48, 9.58. HRMS-ESI (m/z): [M+H]⁺ calcd. for C₁₄H₂₁N₂O 233.1654, found 233.1652.

### Example 3-E. 4-(Cyanomethyl)phenethyl 4-methylbenzenesulfonate (Intermediate 26)

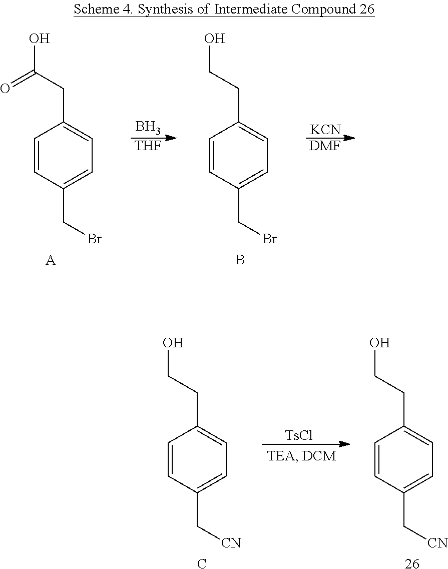

### Example 3-E-1. 2-(4-(Bromomethyl)phenyl)ethan-1-ol (B)

In an argon charged flame dried flask, 2-(4-(bromomethyl)phenyl)acetic acid (A, 5 g, 22 mmol, 1 eq) was dissolved in 67 mL of THF. The solution was cooled to 0° C. and a BH₃/THF complex (1M in THF, 32.7 mmol, 1.5 eq) was added dropwise under an atmosphere of argon, a vigorous initial bubbling occurred. After the bubbling ceased, the reaction mixture was allowed to warm to room temperature and stirred overnight. The reaction mixture was quenched with aq 2N HCl and heated to 50° C. for 2 hours. Volatiles were removed by rotary evaporation and the resulting residue was extracted twice with ethyl acetate. The combined organic phases were washed twice with sodium bicarbonate to remove unreacted carboxylic acid and concentrated to yield 2-(4-(bromomethyl)phenyl)ethan-1-ol (B) as a white solid (3.5 g, 75%). 1H-NMR (400 MHz; DMSO-d6):δ 7.31 (d, J=8.0 Hz, 2H), 7.17 (d, J=8.0 Hz, 2H), 4.64 (s, 2H), 3.56 (t, J=7.0 Hz, 2H), 2.67 (t, J=7.0 Hz, 2H). 13-C NMR (101 MHz; DMSO-d6): δ 140.4, 135.9, 129.61, 129.55, 62.4, 39.1, 35.2.

### Example 3-E-2. 2-(4-(2-Hydroxyethyl)phenyl)acetonitrile (C)

A mixture of 2-(4-(bromomethyl)phenyl)ethan-1-ol (B, 1.85 g, 8.6 mmol, 1 eq) and potassium cyanide (1.68, 25.7 mmol, 3 eq) in DMF (25 mL) was warmed to 70° C. and stirred under an atmosphere of nitrogen overnight. An orange color developed over the course of the reaction. The reaction mixture was cooled to room temperature and quench with a saturated solution of NaHCO₃. The aqueous phase was extracted twice with methyl tert-butyl ester. The combined organic phases were dried with Na₂SO₄ and concentrated to give C as a colorless oil (1.05 g, 76%). ¹H-NMR (CDCl₃): δ 7.27-7.15 (m, 4H), 3.78 (t, J=6.7 Hz, 2H), 3.66 (s, 2H), 2.90 (d, J=5.7 Hz, 1H), 2.81 (t, J=6.3 Hz, 2H). ¹³C NMR (101 MHz; CDCl₃): δ 138.8, 129.7, 128.03, 127.87, 118.0, 63.3, 38.6, 23.2.

### Example 3-E-3. 4-(Cyanomethyl)phenethyl 4-methylbenzenesulfonate (26)

In an argon charged flame dried flask, 2-(4-(2-hydroxyethyl)phenyl)acetonitrile (C, 1 g, 6.2 mmol, 1 eq) was dissolved in 18 mL of dichloromethane. The solution was cooled to 0° C. and p-toluenesulfonyl chloride (1.8 g, 9.2 mmol, 1.5 eq) followed by triethylamine (1.3 g, 12.4 mmol, 2 eq) was added. After allowing the reaction to warm to room temperature it was stirred overnight. Sodium bicarbonate was added to quench the reaction. The aqueous phase was extracted twice with dichloromethane followed by washing the combined organic phases with sodium bicarbonate. Drying and filtering of the organic phase was followed by purification by flash chromatography with 25% EtOAc in hexanes affording 4-(cyanomethyl)phenethyl 4-methylbenzenesulfonate (26, 1.2 g, 62%) as a brown solid. ¹H-NMR (400 MHz; CDCl₃): δ 7.69 (d, J=8.3 Hz, 2H), 7.30 (d, J=8.1 Hz, 2H), 7.22 (d, J=8.0 Hz, 2H), 7.13 (d, J=8.0 Hz, 2H), 4.21 (t, J=6.8 Hz, 2H), 3.71 (s, 2H), 2.96 (t, J=6.8 Hz, 2H), 2.44 (s, 3H). ¹³C NMR (101 MHz; CDCl₃): δ 144.8, 136.4, 132.8, 129.77, 129.65, 128.5, 128.1,

### Example 4-A. 4-(2-Cyanoethyl)phenethyl 4-methylbenzenesulfonate (Intermediate 27)

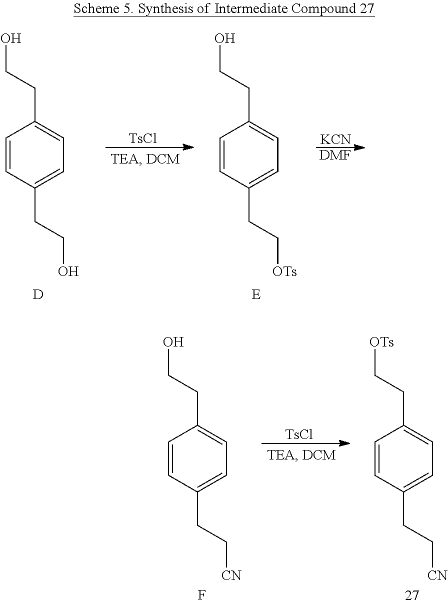

### Example 4-A-1. 4-(2-Hydroxyethyl)phenethyl 4-methylbenzenesulfonate (E)

To a mixture of 1,4-benzenediethanol (D, 5 g, 30 mmol) and TEA (2.3 mL, 16.5 mmol) in anhydrous DCM (70 mL) was added dropwise a solution of p-toluenesulfonyl chloride (2.85 g, 15 mmol) in anhydrous DCM (10 mL) at 0° C. under an atmosphere of nitrogen. The reaction mixture was allowed to warm to room temperature and the stirring was continued overnight. The reaction was quench with water, phases were separated and the aqueous layer extracted with DCM (3×10 mL). The combined organic phases were washed with brine, dried over sodium sulfate, filtered and concd in vacuo. The resulting residue was purified by column chromatography using a gradient of 0-15% EtOAc in DCM yielding 4-(2-hydroxyethyl)phenethyl 4-methylbenzenesulfonate (E) as a colorless oil (2.5 g, 52%). ¹H NMR (CDCl₃) δ 7.69 (d, 2H, J=8.22 Hz), 7.27 (d, 2H, J=8.22 Hz), 7.11 (d, 2H, J=8.02 Hz), 7.04 (d, 2H, J=8.02 Hz), 4.17 (t, 2H, J=7.04 Hz), 3.82 (q, 2H, J=5.87 Hz), 2.91 (t, 2H, J=7.04 Hz), 2.81 (t, 2H, J=7.04 Hz), 2.42 (s, 3H), 1.38 (bs, 1H); ¹³C NMR (CDCl₃): δ 144.6, 137.1, 134.3, 132.9, 129.7, 129.2, 129.1, 127.8, 70.6, 63.6, 38.7, 34.9, 21.6.

### Example 4-A-2. 3-(4-(2-Hydroxyethyl)phenyl)propanenitrile (F)

A mixture of 4-(2-hydroxyethyl)phenethyl 4-methylbenzenesulfonate (E, 2.5 g, 7.8 mmol) and KCN (762 mg, 11.7 mmol) in DMF (10 mL) was heated at 70° C. for 5 hours under an atmosphere of nitrogen until the starting material was consumed as indicated by TLC. The reaction mixture was cooled to room temperature and quenched with ice-cold water (10 mL). The aq layer was extracted with ether (5×15 mL). The combined organic layers were washed with 1N HCl (20 mL), dried over Na₂SO₄ and concd to yield F as a colorless oil (1.23 g, 90%). ¹H NMR (CDCl₃) δ 7.15-7.02 (m, 4H), 3.84 (t, 2H, J=6.65 Hz), 3.82 (t, 2H, J=6.65 Hz), 2.57-2.86 (m, 2H), 2.93 (t, 2H, J=7.434 Hz); ¹³C NMR (CDCl₃): δ 137.5, 136.1, 129.5, 128.4, 119.1, 63.6, 38.7, 31.2, 19.4.

### Example 4-A-3. 4-(2-Cyanoethyl)phenethyl 4-methylbenzenesulfonate (27)

To a mixture of 3-(4-(2-hydroxyethyl)phenyl)propanenitrile (F, 1.23 g, 7.02 mmol) and p-toluenesulfonyl chloride (1.54 g, 8.07 mmol) in anhydrous DCM (210 mL) was added TEA (1.95 mL, 14.04 mmol) in a few portions at 0° C. under an atmosphere of nitrogen. The reaction mixture was allowed to warm to room temperature and the stirring was continued overnight. The reaction was quench with water, phases were separated and the aqueous layer extracted with DCM (3×10 mL). The combined organic phases were washed with brine, dried over sodium sulfate, filtered and concd in vacuo. The resulting residue was purified by column chromatography using a gradient of 0-50% EtOAc in DCM yielding 4-(2-cyanoethyl)phenethyl 4-methylbenzenesulfonate (27) as a colorless oil that solidified upon storage at 4° C. (2.2 g, 95%). ¹H NMR (CDCl₃) δ 7.67 (d, 2H, J=8.21 Hz), 7.27 (d, 2H, J=7.82 Hz), 7.10 (d, 2H, J=8.21 Hz), 7.05 (d, 2H, J=7.82 Hz), 4.16 (t, 2H, J=7.04 Hz), 2.85-2.92 (m, 4H), 2.56 (t, 2H, J=7.04 Hz), 2.39 (s, 3H); ¹³C NMR (CDCl₃): δ 144.8, 136.7, 135.2, 132.2, 129.8, 129.3, 128.5, 127.8, 119.2, 70.5, 34.8, 31.0, 21.6, 19.3.

### Example 3-F. N-(1-(4-Cyanophenethyl)piperidin-4-yl)-N-phenylpropionamide (28)

### Example 4-B. N-(1-(4-(cyanomethyl)phenethyl)piperidin-4-yl)-N-phenylpropionamide (29)

### Example 5-A. N-(1-(4-(2-cyanoethyl)phenethyl)piperidin-4-yl)-N-phenylpropionamide (30)

### General Procedure for Preparing Compounds 28, 29 and 30

To a mixture of N-phenyl-N-(piperidin-4-yl)propionamide (24, 1 eq) and anhydrous K₂CO₃ (powder 325 mesh, 3 eq) in anhydrous acetonitrile (5 mL/mmol of 24) was added a solution of bromo- or tosyl analog of the nitrile (25, 26 or 27, 1.1 eq) in anhydrous acetonitrile (5 mL/mmol of nitrile). The reaction mixture was heated to reflux for 4 hours under an atmosphere of nitrogen, until the starting material was consumed as indicated by TLC. The solution was cooled to room temperature, filtered through a pad of Celite and concentrated in vacuo. The resulting residue (28, 29 or 30, respectively based on nitrile 25, 26 or 27) was purified by column chromatography using a gradient of 0-8% MeOH/5% NH₄OH in CHCl₃.

Compound 28: Obtained from 4-(2-bromoethyl)benzonitrile (25) as a colorless oil (950 mg, 88%). ¹H NMR (CDCl₃) δ 7.50 (d, 2H, J=8.21 Hz), 7.31-7.39 (m, 3H), 7.21-7.24 (m, 2H), 7.03-7.06 (m, 2H), 4.60-4.68 (m, 1H), 2.92 (d, 2H, J=11.44 Hz), 2.72-2.76 (m, 2H), 2.48-2.52 (m, 2H), 2.13 (dt, 2H, J=12.03 and 2.05 Hz), 1.89 (q, 2H, J=7.48 Hz), 1.76-1.77 (m, 2H), 1.32-1.42 (m, 2H), 0.98 (t, 3H, J=7.48 Hz); ¹³C NMR (CDCl₃): δ 173.5, 145.9, 138.8, 132.1, 130.3, 129.4, 129.3, 128.3, 118.9, 109.9, 59.5, 53.0, 52.0, 33.9, 30.5, 28.5, 9.6; HRMS-ESI (m/z): [M+H]⁺ calcd for C₂₃H₂₈N₃O: 362.2232; found: 362.2228.

Compound 29: Obtained from 4-(cyanomethyl)phenethyl 4-methylbenzenesulfonate (26) as a light yellow syrup (0.765 g, 95%). ¹H-NMR (400 MHz; CDCl₃): δ 7.42-7.07 (m, 9H), 4.68 (tt, J=12.2, 3.9 Hz, 1H), 3.71 (d, J=3.3 Hz, 2H), 2.96 (dd, J=15.8, 9.1 Hz, 2H), 2.72 (dd, J=10.1, 6.3 Hz, 2H), 2.53-2.49 (m, 2H), 2.18-2.13 (m, 2H), 1.93 (q, J=7.4 Hz, 2H), 1.81 (d, J=12.0 Hz, 2H), 1.42 (qd, J=12.3, 3.6 Hz, 2H), 1.01 (t, J=7.4 Hz, 3H). ¹³C NMR (101 MHz; CDCl₃): δ 173.4, 144.7, 140.2, 138.7, 136.2, 132.7, 130.3, 129.69, 129.66, 129.55, 129.26, 129.19, 128.16, 128.04, 127.84, 127.69, 127.4, 117.9, 70.1, 63.2, 60.1, 53.0, 52.0, 38.6, 34.8, 33.2, 30.4, 28.4, 23.14, 23.11, 21.5, 9.5.

Compound 30: Obtained from 4-(2-cyanoethyl)phenethyl 4-methylbenzenesulfonate (27) as a light-yellow oil (919 mg, 91%). ¹H NMR (CDCl₃) δ 7.33-7.39 (m, 3H), 7.05-7.10 (m, 6H), 4.63-4.69 (m, 1H), 2.96 (d, 2H, J=11.74 Hz), 2.89 (t, 2H, J=7.43 Hz), 2.67-2.71 (m, 2H), 2.56 (t, 2H, J=7.43 Hz), 2.47-2.52 (m, 2H), 2.13 (t, 2H, J=11.73 Hz), 1.90 (q, 2H, J=7.48 Hz), 1.77-1.80 (m, 2H), 1.40 (dt, 2H, J=12.52 and 3.91 Hz), 0.99 (t, 3H, J=7.48 Hz; ¹³C NMR (CDCl₃): δ 173.5, 139.2, 138.8, 135.7, 130.4, 129.2, 129.1, 128.2, 128.2, 119.1, 60.4, 53.1, 52.1, 33.4, 31.1, 30.5, 28.5, 19.4, 9.6; HRMS-ESI (m/z): [M+H]⁺ calcd for C₂₅H₃₂N₃O: 390.2545; found: 390.2540.

### Example 3-G. N-(1-(4-(Aminomethyl)phenethyl)piperidin-4-yl)-N-phenylpropionamide (31)

### Example 4-C. N-(1-(4-(2-Aminoethyl)phenethyl)piperidin-4-yl)-N-phenylpropionamide (32)

### Example 5-B. N-(1-(4-(3-Aminopropyl)phenethyl)piperidin-4-yl)-N-phenylpropionamide (33)

### General Procedure for Preparing Compounds 31, 32 and 33

To a solution of the respective alkylation product (28, 29 or 30, 1 eq) in EtOH (18 mL/mmol of nitrile) in a 250 mL pressure tested reaction bottle was added conc. HCl (6 eq) followed by Escat 103 5% Pd/C (0.5 eq w/w) and the vessel was pressurized to 40 psi H2 in a Parr shaker. The reaction mixture was shaken for 48 h, until starting material was consumed as indicated by TLC. The reaction mixture was filtered through Celite, and concentrated in vacuo. The resulting residue was taken in aq 5% NaOH and the aqueous layer was extracted with CHCl₃. The combined organic extracts were dried over Na₂SO₄, and concd in vacuo. The crude product (31, 32 or 33, respectively based on alkylation product 28, 29 or 30) was used in the next step of the synthesis unless indicated otherwise.

Compound 31: The crude product (a light-yellow oil (300 mg, 80%) was used in the next step of the synthesis. ¹H NMR (CDCl₃) δ 7.34-7.39 (m, 3H), 7.18 (d, 2H, J=7.83), 7.10 (d, 2H, J=7.83), 7.05-7.07 (m, 2H), 4.63-4.71 (m, 1H), 3.79 (s, 2H), 2.97-3.00 (m, 2H), 2.68-2.73 (m, 2H), 2.49-2.53 (m, 2H), 2.15 (t, 2H, J=10.95 Hz), 1.90 (q, 2H, J=7.44 Hz), 1.38-1.47 (m, 2H), 0.99 (t, 3H, J=7.44 Hz); ¹³C NMR (CDCl₃): δ 173.5, 141.1, 138.8, 138.6, 130.4, 129.4, 128.8, 128.2, 127.1, 60.4, 53.0, 52.0, 46.2, 33.3, 30.4, 28.5, 9.6; HRMS-ESI (m/z): [M+H]⁺ calcd for C₂₃H₃₂N₃O: 366.2545; found: 366.2552; Anal calcd for C₂₃H₃₁N₃O×0.2 EtOAc: C, 74.61; H, 8.58; N, 10.97; found: C, 74.27; H, 8.22; N, 11.25.

Compound 32: The crude product was purified by column chromatography using a gradient of 0-20% MeOH/5% NH₄OH in CHCl₃ to yield 0.06 g, 60%. ¹H NMR (CDCl₃): δ 7.41-7.35 (m, 3H), 7.14-7.06 (m, 6H), 4.72-4.66 (m, 1H), 2.98 (t, J=13.0 Hz, 2H), 2.93 (t, J=6.7 Hz, 2H), 2.72-2.68 (m, 4H), 2.54-2.50 (m, 2H), 2.18-2.12 (m, 2H), 1.93 (q, J=7.4 Hz, 2H), 1.81 (d, J=12.0 Hz, 2H), 1.43 (qd, J=12.3, 3.6 Hz, 2H), 1.32 (s, 2H), 1.02 (t, J=7.4 Hz, 3H). 5 ¹³C NMR (101 MHz; CDCl₃): δ; δ 173.5, 138.8, 138.0, 137.4, 130.4, 129.2, 128.99, 128.80, 128.66, 128.2, 60.5, 53.1, 52.1, 43.5, 39.6, 33.4, 30.5, 28.5, 9.6; HRMS: [C₂₄H₃₃N₃O]H⁺ Calculated: 380.2702, Observed: 380.2697.

Compound 33: The crude product (a light-yellow oil (681 mg, 97%) was used in the next step of the synthesis. ¹H NMR (CDCl₃) δ 7.31-7.39 (m, 3H), 7.03-7.07 (m, 6H), 4.70-4.62 (m, 1H), 2.97 (d, 2H, J=11.73 Hz), 2.65-2.69 (m, 2H), 2.58 (t, 2H, J=8.22 Hz), 2.47-2.52 (m, 2H), 2.13 (t, 2H, J=7.43 Hz), 1.90 (q, 2H, J=7.44 Hz), 1.68-1.78 (m, 4H), 1.36-1.49 (m, 5H), 0.99 (t, 3H, J=7.43 Hz); ¹³C NMR (CDCl₃): δ 173.5, 139.8, 138.8, 137.5, 130.4, 129.2, 128.6, 128.3, 128.2, 60.6, 53.1, 52.1, 41.8, 35.4, 33.4, 32.8, 30.5, 28.5, 9.6; HRMS-ESI (m/z): [M+H]⁺ calcd for C₂₅H₃₆N₃O: 394.2858; found: 394.2855; Anal calcd for C₂₅H₃₅N₃O×2HCl×0.75H₂O: C, 62.56; H, 8.08; N, 8.75; found: C, 62.66; H, 8.11; N, 8.64.

### Example 3-H. N-Phenyl-N-(1-(4-((3-(tritylthio)propanamido)methyl)phenethyl)piperidin-4-yl)propionamide (34, trityl capped para-AmMeFenHap)

### Example 4-D. N-Phenyl-N-(1-(4-(2-(3-(tritylthio)propanamido)ethyl)phenethyl)piperidin-4-yl)propionamide (35, trityl capped para-AmEtFenHap)

### Example 5-C. N-Phenyl-N-(1-(4-(3-(3-(tritylthio)propanamido)propyl)phenethyl)piperidin-4-yl)propionamide (36, trityl capped para-AmPrFenHap)

### General Procedure for Preparing Compounds 34, 35 and 36

To a mixture of 3-thiotrityl propionic acid (1.1 eq) and HATU (1.5 eq) in anhydrous DCM (6 mL/mmol of acid) was added TEA (3 eq) at 0° C. under an argon atmosphere. The mixture was stirred for 0.5 hour and a solution of the respective amine (31, 32 or 33, 1 eq) in anhydrous DCM (6 mL/mmol of amine) was added in a few portions. The reaction mixture was allowed to warm to room temperature and the stirring was continued for additional hour. The reaction was quench with aq 1N NaOH, phases were separated and the aqueous phase extracted with DCM. The combined organic phases were washed twice with brine, dried over sodium sulfate, filtered and concd in vacuo. The resulting residue was purified twice by column chromatography using a gradient of 1-8% MeOH/5% NH₄OH in CHCl₃.

Compound 34 (trityl capped para-AmMeFenHap): Obtained in the reaction with N-(1-(4-(aminomethyl)phenethyl)piperidin-4-yl)-N-phenylpropionamide (31) as a white foam, 910 mg (70%). ¹H NMR (CDCl₃) δ 7.04-7.39 (m, 24H), 5.72 (bs, 1H), 4.62-4.69 (m, 1H), 4.27 (d, 2H, J=5.87 Hz), 2.95 (d, 2H, J=11.25 Hz), 2.65-2.69 (m, 2H), 2.45-2.51 (m, 4H), 2.12 (t, 2H, J=11.73 Hz), 2.02 (t, 2H, J=7.34 Hz), 1.92 (q, 2H, J=7.34 Hz), 1.78 (d, 2H, J=11.73 Hz), 1.39 (dt, 2H, J=12.22 and 3.42 Hz), 0.99 (t, 3H, J=7.34 Hz); ¹³C NMR (CDCl₃): δ 173.5, 170.7, 144.6, 139.5, 138.8, 135.8, 130.4, 129.5, 129.3, 128.9, 128.2, 127.9, 126.6, 66.8, 60.4, 53.1, 52.1, 43.2, 35.6, 33.4, 30.5, 28.5, 27.7, 9.6; HRMS-ESI (m/z): [M+H]⁺ calcd for C₄₅H₅₀N₃O2S: 696.3624; found: 696.3633; Anal calcd for C₄₅H₄₉N₃O₂S×0.5 DCM: C, 74.01; H, 6.82; N, 5.69; found: C, 73.88; H, 6.79; N, 5.79.

Compound 35 (trityl capped para-AmEtFenHap): Obtained in the reaction with N-(1-(4-(2-aminoethyl)phenethyl)piperidin-4-yl)-N-phenylpropionamide (32) as a white foam 0.113 g (26%). ¹H-NMR (400 MHz; CDCl₃): δ 7.42-7.32 (m, 8H), 7.26-7.23 (m, 7H), 7.20-7.16 (m, 3H), 7.09-6.98 (m, 6H), 5.30-5.22 (m, 1H), 4.69 (tt, J=12.2, 3.8 Hz, 1H), 3.44-3.35 (m, 2H), 3.04-2.95 (m, 2H), 2.74-2.63 (m, 4H), 2.56-2.45 (m, 4H), 2.21-2.09 (m, 2H), 1.93 (q, J=7.4 Hz, 2H), 1.88 (q, J=6.9 Hz, 2H), 1.79 (t, J=12.4 Hz, 2H), 1.50-1.36 (m, 2H), 1.06-0.98 (m, 3H). ¹³C NMR (101 MHz; CDCl₃): δ 173.5, 170.8, 144.6, 138.8, 138.4, 136.4, 130.4, 129.2, 128.81, 128.78, 128.2, 126.6, 66.8, 60.4, 53.1, 52.1, 40.5, 35.7, 35.1, 33.3, 30.5, 28.5, 27.7, 9.6. HRMS: [C₄₆H₅₁N₃O₂S]H⁺ Calculated: 710.3780, Observed: 710.3785. C₄₆H₅₁N₃O₂S×0.15 CHCl₃×0.05 H₂O: C, 76.06; H, 7.09; N, 5.77; found: C, 77.48; H, 7.46; N, 5.68.

Compound 36 (trityl capped para-AmPrFenHap): Obtained in the reaction with N-(1-(4-(3-aminopropyl)phenethyl)piperidin-4-yl)-N-phenylpropionamide (33) as a white foam, 764 mg (84%). ¹H NMR (CDCl₃) δ 6.97-7.40 (m, 24H), 5.21 (bs, 1H), 4.64-4.69 (m, 1H), 3.16 (q, 2H, J=6.36 Hz), 2.97 (d, 2H, J=11.73 Hz), 2.64-2.68 (m, 2H), 2.54 (t, 2H, J=7.34 Hz), 2.44-2.50 (m, 4H), 2.09-2.15 (m, 2H), 1.88-1.96 (m, 4H), 1.68-1.79 (m, 4H), 1.36-1.45 (m, 2H), 0.99 (t, 3H, J=7.34 Hz); ¹³C NMR (CDCl₃): δ 173.5, 170.8, 144.6, 139.0, 138.8, 137.8, 130.4, 129.5, 129.2, 128.7, 128.3, 128.2, 127.9, 126.6, 66.8, 60.5, 53.1, 52.1, 39.1, 35.7, 33.4, 32.7, 31.1, 30.5, 28.5, 27.8, 9.6; HRMS-ESI (m/z): [M+H]⁺ calcd for C₄₇H₅₄N₃O₂S: 724.3937; found: 724.3932; Anal calcd for C₄₇H₅₃N₃O₂S×0.3H₂O: C, 77.39; H, 7.41; N, 5.76; found: C, 77.48; H, 7.46; N, 5.68.

### Example 6. N-Phenyl-N-(1-(2-(3-(tritylthio)propanamido)phenethyl)piperidin yl)propionamide (37, Trityl Capped Ortho-AmFenHap)

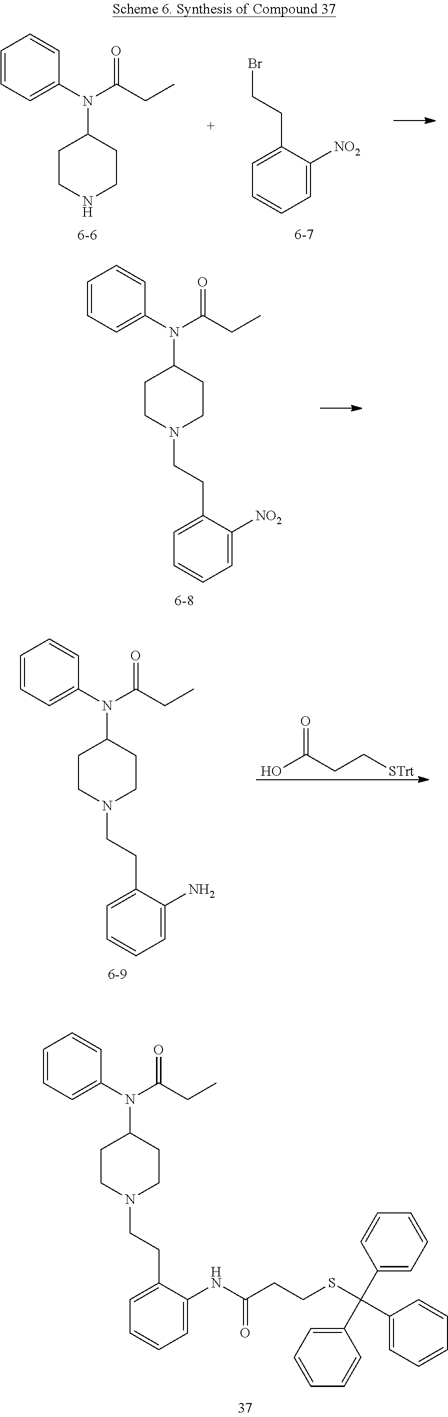

N-(1-(2-Nitrophenethyl)piperidine-4-yl)-N-phenylpropionamide (6-8). A solution of 2-nitrophenethyl bromide (6-7) (316 mg, 1.373 mmol, 1.1 equiv) in dry acetonitrile (6.0 mL) was added dropwise to a solution of norfentanyl (6-6) (290 mg, 1.248 mmol, 1.0 equiv.) and K₂CO₃ (517 mg) in dry acetonitrile (6.5 mL). This solution was stirred under argon for 3 h and additional nitrophenethyl bromide (6-7) (1.4 equiv.) was added before leaving the reaction to stir under argon for 16 h. The reaction mixture was filtered through a pad of celite and concentrated. The crude mixture was purified via flash chromatography eluting with 0-10% 80:19:1 CHCl₃:MeOH:NH₄OH in CHCl₃. The product (6-8) was obtained as a colorless foam (297 mg, 62%). Analytically pure material was obtained by crystallization from cyclohexane to yield colorless needles. (mp 114-115° C.) ¹H NMR (400 MHz; CDCl₃): δ 7.85 (d, J=7.6 Hz, 1H), 7.48 (t, J=7.5 Hz, 1H), 7.42-7.30 (m, 5H), 7.10-7.08 (m, 2H), 4.67 (tt, 0.1=12.1, 3.8 Hz, 1H), 2.91-3.06 (m, 4H), 2.61-2.57 (m, 2H), 2.23 (t, J=11.1 Hz, 2H), 1.93 (q, J=7.4 Hz, 2H), 1.80 (d, J=12.0 Hz, 2H), 1.41 (qd, J=12.2, 3.6 Hz, 2H), 1.02 (t, J=7.4 Hz, 3H). ¹³C NMR (100 MHz; CDCl₃): δ 173.5, 149.6, 139.0, 135.4, 132.9, 132.3, 130.6, 129.4, 128.3, 127.2, 124.7, 59.0, 53.0, 52.2, 30.8, 30.6, 28.5, 9.7; HRMS-ESI (m/z): [M+H]₊ calcd for C₂₂H₂₈N₃O₃: 382.2131; found: 382.2124.

N-(1-(2-Aminophenethyl)piperidine-4-yl)-N-phenylpropionamide (6-9). A solution of 6-8 (228 mg, 0.598 mmol, 1.0 equiv.) in EtOH (20 mL) was transferred to a pressure bottle, Escat 103 (5% Pd/C, 0.05 g) was added and the bottle pressurized to 35 psi Hz in a Parr shaker. After 16 h, the reaction was filtered through celite and concentrated under vacuum to yield a brown oil which was used crude in the following conjugation reaction. Analytically pure material was obtained by crystallization from toluene to yield light brown crystals. (mp 128-129° C.) ¹H-NMR (400 MHz; CDCl₃): δ 7.39 (d, J=7.4 Hz, 3H), 7.08 (d, J=6.7 Hz, 2H), 6.98 (dd, 0.1=16.8, 7.8 Hz, 2H), 6.69-6.60 (m, 2H), 4.70-4.63 (m, 1H), 3.99 (s, 2H), 3.01 (d, J=11.4 Hz, 2H), 2.62 (t, J=7.0 Hz, 2H), 2.52 (t, J=7.0 Hz, 2H), 2.15 (t, J=11.5 Hz, 2H), 1.92 (q, 0.1=7.4 Hz, 2H), 1.80 (d, J=11.4 Hz, 2H), 1.39 (qd, J=12.4, 3.2), 1.01 (t, J=7.4 Hz, 3H). ¹³C NMR (100 MHz; CDCl₃): δ 173.6, 145.1, 139.1, 130.51, 130.45, 130.2, 129.43, 129.38, 128.4, 127.4, 125.7, 118.7, 115.8, 58.6, 53.5, 52.3, 30.8, 30.3, 28.7, 9.7; HRMS-ESI (m/z): [M+H]₊ calcd for C₂₂H₃₀N₃O: 352.2389; found: 382.2124.

N-Phenyl-N-(1-(2-(3-(tritylthio)propanamido)phenethyl)piperidin-4-yl)propionamide (37). To a solution of 3-mercaptotrityl-propanoic acid (250 mg, 0.7176 mmol, 1.2 equiv.) in dry DCM (3.5 mL) was added HATU (273, 0.7176 mmol, 1.2 equiv). This solution was cooled to 0° C. and triethylamine was added. After 30 min, a solution of crude 6-9 (0.598 mmol, 1.0 equiv.) in dry DCM (3.5 mL) was added dropwise. After 10 min the reaction was allowed to warm to rt and stirred under argon for 16 h. The mixture was transferred to a separatory funnel and washed with 1 M KOH (30 mL), brine (15 mL), and dried over MgSO₄. The crude residue was purified via flash chromatography eluting with 2-8% 50:45:5 CHCl₃:MeOH:NH₄OH in CHCl₃ to yield 37. Analytically pure material was obtained through recrystallization from cyclohexane:toluene (2:1). (mp 188-189° C.) ¹H-NMR (400 MHz; CDCl₃): δ 9.99 (s, 1H), 7.83 (d, J=8.1 Hz, 1H), 7.42 (t, J=9.0 Hz, 6H), 7.30-7.13 (m, 13H), 7.00 (quintet, 0.1=7.6 Hz, 4H), 4.70-4.62 (m, 1H), 2.86 (d, J=11.8 Hz, 2H), 2.62 (t, J=5.1 Hz, 2H), 2.51 (t, J=4.9 Hz, 2H), 2.35 (t, J=7.0 Hz, 2H), 2.17 (t, J=11.5 Hz, 2H), 1.82-1.93 (m, 4H), 1.76-1.73 (m, 2H), 1.34-1.28 (m, 2H), 1.00 (t, J=7.4 Hz, 3H). ¹³C NMR (100 MHz; CDCl₃): δ 173.6, 169.2, 144.9, 138.7, 136.6, 133.3, 130.4, 130.1, 129.70, 129.59, 128.6, 128.0, 127.0, 126.8, 124.6, 123.6, 66.7, 60.7, 54.5, 51.6, 36.0, 32.0, 30.7, 28.6, 27.8, 9.7; HRMS-ESI (m/z): [M+H]₊ calcd for C₄₄H₄₈N₃O₂S: 682.3467; found: 682.3454.

### Example 7. N-Phenyl-N-(1-(3-(3-(tritylthio)propanamido)phenethyl)piperidin-4-yl)propionamide (38, Trityl Capped Meta-AmFenHap)

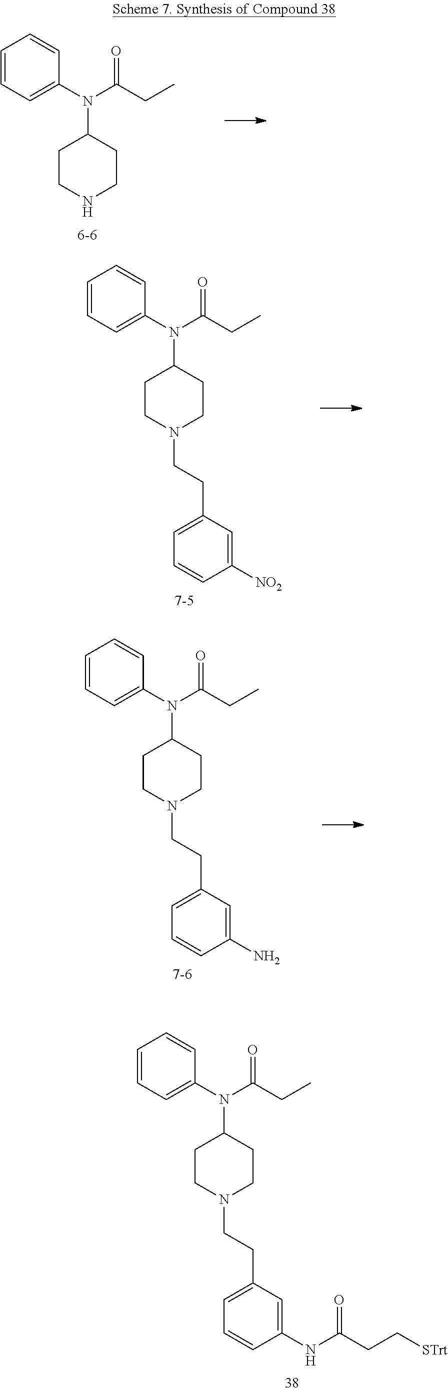

N-(1-(3-Nitrophenethyl)piperidin-4-yl)-N-phenylpropionamide (7-5). To N-phenyl-N-(piperidin-4-yl)propionamide (264 mg, 1.14 mmol) and K₂CO₃ (469 mg, 3.41 mmol) in an oven dried flask was added 5.7 mL acetonitrile. The reaction mixture was treated with 3-nitrophenethyl bromide (287 mg, 1.25 mmol) in 6.25 mL acetonitrile and stirred at reflux at 4.5 h. Upon completion, the reaction mixture was cooled and filtered through celite. Purification by flash column chromatography on silica gel (0-8% CMA in CHCl₃) to afford a yellow oil. Crystallization from cyclohexane afforded white crystals (0.34 g, 79% yield). mp 102-104° C. ¹H NMR (400 MHz; CDDl₃): d 8.04-8.02 (m, 2H), 7.47 (d, J=7.8 Hz, 1H), 7.43-7.35 (m, 4H), 7.08 (dt, J=6.4, 2.0 Hz, 2H), 4.72-4.64 (m, 1H), 2.98-2.95 (m, 2H), 2.82 (dd, J=9.3, 6.6 Hz, 2H), 2.57 (dd, J=9.3, 6.7 Hz, 2H), 2.22-2.15 (m, 2H), 1.92 (q, J=7.2 2H), 1.83-1.79 (m, 2H), 1.46-1.36 (m, 2H), 1.02 (t, J=7.2 Hz, 3H). ¹³C NMR (101 MHz; CDCl₃): δ 173.6, 148.4, 142.5, 139.0, 135.0, 130.5, 129.43, 129.30, 128.4, 123.6, 121.3, 59.7, 53.2, 52.2, 33.5, 30.7, 28.6, 9.7. HRMS-ESI (m/z): [M+H⁺] calcd for C₂₂H₂₈N₃O₃: 382.2131; found: 382.2128.

N-(1-(3-Aminophenethyl)piperidin-4-yl)-N-phenylpropionamide (7-6). A solution of 7-5 (286 mg, 0.75 mmol) in 15 mL ethanol was transferred to a pressure tested vessel. Escat 103 (5% Pd/C, 15 mg) and HCl (103 mL, 1.50 mmol) were added to vessel and the bottle was pressurized to 30 psi H2 in a Par shaker. After 17 h, the reaction was filtered through celite and concentrated to afford an analytically pure brown oil as the HCl salt. For analysis the residue was taken up in 5% NaOH and extracted with CHCl₃. The combined organic extracts were dried over Na₂SO₄ and concentrated to afford the free base as a yellow oil (248 mg, 94% yield). ¹H NMR (400 MHz; CDCl₃): δ 7.41-7.33 (m, 3H), 7.09-7.02 (m, 3H), 6.56-6.49 (m, 3H), 4.69 (tt, J=12.2, 3.8 Hz, 1H), 3.65-3.54 (br s, 2H), 2.99 (d, J=11.4 Hz, 2H), 2.64 (dd, J=11.4, 5.2 Hz, 2H), 2.54-2.50 (m, 2H), 2.15 (t, J=11.2 Hz, 2H), 1.93 (q, J=7.4 Hz, 2H), 1.80 (d, J=12.0 Hz, 2H), 1.48-1.38 (m, 2H), 1.02 (t, J=7.4 Hz, 3H). ¹³C NMR (101 MHz; CDCl₃): δ 173.6, 146.5, 141.6, 139.0, 130.6, 129.4, 128.3, 119.0, 115.5, 113.0, 60.5, 53.2, 52.3, 33.9, 30.7, 28.6, 9.7. HRMS-ESI (m/z): [M+H⁺] calcd for C₂₂H₃₀N₃O: 352.2389; found: 352.2384.

N-Phenyl-N-(1-(3-(3-(tritylthio)propanamido)phenethyl)piperidin-4-yl)propionamide (38). To a solution of 3-thiotritylpropionic acid and HATU in 4 mL DCM at 0° C. under an argon atmosphere was added TEA. The mixture was stirred for 30 min whereupon 7-6 (211 mg, 0.6 mmol) in 4 mL DCM was added. The mixture was allowed to warm to room temperature and was stirred for 2 h. The reaction was quenched with 1N NaOH and the aqueous phase was extracted with DCM. The combined organic phases were washed with brine and dried over Na₂SO₄ and concentrated. Purification by flash column chromatography on silica gel (1%-8% CMA in CHCl₃) to afford compound 38. ¹H-NMR (400 MHz; CDCl₃): δ 7.42 (d, J=7.8 Hz, 6H), 7.41-7.31 (m, 3H), 7.28-7.23 (m, 6H), 7.23-7.13 (m, 5H), 7.07-7.04 (m, 2H), 7.03 (br s, 1H), 6.86 (d, J=7.1 Hz, 1H), 4.69-4.61 (m, 1H), 2.94 (d, J=11.7 Hz, 2H), 2.69-2.65 (m, 2H), 2.57 (t, J=7.3 Hz, 2H), 2.51-2.47 (m, 2H), 2.14-2.09 (m, 4H), 1.91 (q, J=7.4 Hz, 2H), 1.79-1.72 (m, 2H), 1.45-1.35 (m, 2H), 1.00 (t, J=7.4 Hz, 3H). ¹³C NMR (101 MHz; CDCl₃): δ 173.7, 169.2, 144.8, 141.4, 139.0, 137.9, 130.6, 129.7, 129.4, 129.0, 128.4, 128.1, 126.9, 124.8, 120.1, 117.6, 67.1, 60.4, 53.2, 52.3, 37.0, 33.9, 30.7, 28.7, 27.8, 9.8. HRMS-ESI (m/z): [M+H⁺] calcd for C₄₄H₄₇N₃O₂S: 682.3467; found: 382.3439.

### Deprotection of Trityl-Capped Haptens

Trityl-capped haptens as described herein can be solubilized in chloroform (1.5 mL), treated with trifluoroacetic acid (150 μL) and triethylsilane (75 μL) for 1 h at room temperature, and concentrated under vacuum overnight. The residue can be washed with petroleum ether and evaporated to dryness under vacuum. The residue can be reconstituted in dimethyl sulfoxide (DMSO) (1 mL) and used for subsequent conjugation.

### Example 8

Compound 19 was deprotected to afford para-AmFenHap as follows. Briefly, Compound 19 (12 mg) was solubilized in chloroform (1.5 mL), treated with trifluoroacetic acid (150 μL) and triethylsilane (75 μL) for 1 h at room temperature, and concentrated under vacuum overnight. The residue was washed with petroleum ether and evaporated to dryness under vacuum. The residue was reconstituted in dimethyl sulfoxide (DMSO) (1 mL) and used for subsequent conjugation.

Compound 14 was deprotected according to the deprotection methods described herein to afford “Am-FenHap1” having the following structure:

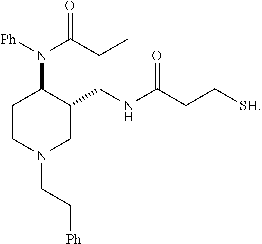

### Protocol for Preparing Carrier-Fentanyl Hapten Conjugate

The amount of hapten that is conjugated to the carrier regulates the immune response induced by the hapten (Jalah, R. et al., Bioconjugate Chemistry (2015) 26:1041-1053). Various strategies which are known in the art can be used in accordance with the disclosure to optimize the amount of conjugated hapten. For example, the extent of derivatization of the protein with cross-linker can be influenced by varying experimental conditions such as the concentration of each of the reaction partners, the excess of one reagent over the other, the pH, and the temperature. In general, the ratio of hapten to carrier is dependent upon the number of accessible lysines on the carrier and the amount of hapten added to the reaction.

### Example 9. Preparation of Tetanus-Toxoid-Fentanyl Hapten Conjugate

(1) Dialyzed tetanus toxoid (TT) in phosphate buffered saline (pH 7.2). (2) Placed 1165 μL of TT (1461 μg/mL) in a reaction vessel and subsequently added 71.4 μL of SM (PEG)₂ linker (250 mM in DMSO). The molar ratio of linker to protein was 1600:1. (3) Incubated for 2 hrs at room temperature. (4) Passed the reaction mixture through a desalting column to remove excess succinimidyl-[(N-maleimido-propionamido)-diethylene glycol]ester (SM(PEG)₂) linker. (5) Determined the protein concentration of π-linker. (6) Placed 718 μL of π-linker (964 μg/mL) in a reaction vessel and subsequently added 115 of the fentanyl hapten (16.5 mM in DMSO). The trityl protecting group of the fentanyl hapten was previously removed using 10% trifluoroacetic acid in chloroform. The molar ratio of hapten to protein was 300:1. (7). Incubated for 2 hrs at room temperature. (8) Placed the reaction mixture in a dialysis cassette and dialyzed overnight at 4° C. against phosphate buffered saline (pH 7.4) to remove excess fentanyl hapten. (9) Determined the protein concentration of the TT-fentanyl hapten conjugate. (10) Measured the amount of hapten conjugated to TT using matrix-assisted laser desorption/ionization time-of-flight (MALDI TOF) mass spectrometry (MS).

Para-AmFenHap was conjugated to TT (to form “π-para-AmFenHap” or “TT-paraFenHap conjugate”) as follows. Briefly, surface amino groups in TT (1 mg/mL stock) were activated by reacting with a solution of 250 mM SM(PEG)₂ in DMSO at a protein/linker ratio of 1:1600 for 2 h at 25° C. in BupH 7.2 (100 mM sodium phosphate, 150 mM sodium chloride, pH 7.2). Excess linker was removed by a spin column (Zeba, 7k MWCO), and the flow through containing π-maleimide was reacted with deprotected para-AmFenHap at a protein/hapten molar ratio of 1:300 for 2 h at 25° C. in BupH 7.2. Before being used for conjugation, the hapten concentration was measured by Ellman's assay, where about 20-30 mM was obtained. The reaction products were transferred to dialysis cassettes (Slide-A-Lyzer G2, 10k MWCO) and repeatedly dialyzed overnight against DPBS, pH 7.4 at 4° C. Protein concentration was quantified using Pierce BCA assay kit following manufacturer's instructions.

### Example 10. Preparation of Bovine Serum Albumin-Fentanyl Hapten Conjugate

(1) Reconstituted lyophilized Bovine Serum Albumin (BSA) with water. The excipient of lyophilized BSA was phosphate buffered saline (pH 7.2). (2) Placed 250 μL of BSA (10 mg/mL) in a reaction vessel and subsequently added 1.5 μL of SM (PEG)₂ linker (250 mM in DMSO). The molar ratio of linker to protein was 10:1. (3) Incubated for 2 hrs at room temperature. (4) Passed the reaction mixture through a desalting column to remove excess SM(PEG)₂ linker. (5) Determined the protein concentration of BSA-linker. (6) Placed 268 of π-linker (7.5 mg/mL) in a reaction vessel and subsequently added 31.5 μL of fentanyl hapten (23.9 mM in DMSO). The trityl protecting group of the fentanyl hapten was previously removed using 10% trifluoroacetic acid in chloroform. The molar ratio of hapten to protein was 25:1. (7). Incubated for 2 hrs at room temperature. (8) Placed the reaction mixture in a dialysis cassette and dialyzed overnight at 4° C. against phosphate buffered saline (pH 7.4) to remove excess fentanyl hapten. (9) Determined the protein concentration of BSA-fentanyl hapten conjugate. (10) Measured the amount of hapten conjugated to BSA using MALDI TOF MS.

BSA-para-AmFenHap was prepared as follows. Briefly, BSA was reacted with 250 mM SM(PEG)₂ (1:10 molar ratio) at RT for 2 h. Excess linker was removed via spin column filtration (Zeba™, 7K MWCO, Thermo Fisher Scientific, Rockford, Ill.) and the eluate containing BSA-maleimide was reacted with deprotected hapten (1:300 protein:hapten molar ratio) at CC for 2 h. The reaction was transferred to a dialysis cassette (Slide-A-Lyzer™, 10 kDa MWCO) and dialyzed overnight against DPBS pH 7.4 to remove unreacted hapten. After sterile-filtration using 0.2 μM polypropylene filter (Whatman), protein concentration and hapten density were measured using BCA assay and MALDI-TOF mass spectrometry, respectively, according to methods known in the art.

### Example 11. Determination of Hapten Density

Hapten density was quantified by matrix-assisted laser desorption/ionization time-of-flight MS (MALDI-TOF MS), according to methods known in the art. Briefly, unconjugated TT, unconjugated BSA, π-para-AmFenHap, and BSA-para-AmFenHap were desalted using C4 ZipTip. Samples (0.5 μL) were mixed with (0.5 μL) sinapinic acid (10 mg/mL) in 50:50 ACN/H₂O 0.1% formic acid (FA) and spotted on a MALDI-TOF 384-well stainless plate and loaded to the AXIMA MegaTOF instrument (Shimadzu Scientific Instruments, Columbia, Md.). The instrument was calibrated using either IgG (for samples containing TT) or BSA (for samples containing BSA). MS were acquired using the following settings: tuning mode, linear; laser power, 60-70; profiles, 500; shots, 2 per profile. Spectra were smoothed using the Gaussian method, and masses were assigned using threshold apex peak detection method. The number of the haptens attached per protein molecule was calculated using the following equation:

hapten density=(mass_{protein-hapten conjugate}−mass_{unconjugated protein})/massₗᵢₙₖₑᵣ₊ₕₐₚₜₑₙ

The net addition mass for linker+hapten, massₗᵢₙₖₑᵣ₊ₕₐₚₜₑₙ=749.74 g/mol.

MALDI-TOF MS spectra of π-free: [M+H]⁺=164,203.10; MALDI-TOF MS spectra of π-para-AnnFenHap conjugate (about 31-32 haptens): [M+H]⁺=187,627.32.

MALDI-TOF MS spectra of BSA-free: [M+H]⁺=66,416.35; MALDI-TOF MS spectra of BSA-para-AmFenHap conjugate (about 6-7 haptens): [M+H]⁺=71,621.12.

### Vaccine Formulation & Biological Studies

### Example 12

The final vaccine formulation (50 μL) was composed of 10 μg of π-para-AnnFenHap (based on the protein content of the protein-hapten conjugate), 20 μg of synthetic monophosphoryl 3-deacyl lipid A (3D-PHAD) in ALF43, and 30 μg of aluminum in aluminum hydroxide (Alhydrogel) in DPBS pH 7.4. ALF43 contained DMPC/DMPG/cholesterol/3D-PHAD at a molar ratio of 9:1:7.5:1.136; the molar ratio of phospholipids/3D-PHAD was 8.8:1. ALF43, derived from small unilamellar vesicles, was prepared as lyophilized powder following methods known in the art. The total concentration of phospholipids in the reconstituted ALF43A was 2.29 mM.

Briefly, ˜7-week-old female BALB/c mice (n=10 control and n=10 vaccine group) (Jackson Laboratories, Bar Harbor, Me.) were immunized via intramuscular (i.m.) route at alternate rear thighs with 50 μL of vaccine formulation on weeks 0, 3, 6, and 14. Challenge experiments were performed at weeks 18 and 22 via a subcutaneous (s.c.) route using fentanyl.HCl in 0.9% saline (0.0050 to 4.0 mg/kg). This route has been used previously to evaluate anti-fentanyl vaccines. Control mice did not receive any vaccination. Antinociceptive effects were assessed 15 min after each fentanyl injection.

Two nociception assays, tail immersion and hot plate, were used to evaluate vaccine efficacy. In the tail-immersion assay, the mouse tail was immersed in a water bath set at 54° C. (IITC Life Science, Woodland Hills, Calif.). The latency times were measured with a cutoff time of 8 s to prevent tail injury. Antinociception, measured as % maximum potential effect (% MPE), was calculated as follows:

% MPE=(post fentantyl injection latency time−baseline latency time)/(cutoff latency−baseline latency time)*100

In the hot plate assay, the mouse was placed on a hot plate analgesia meter (Harvard Apparatus, Holliston, Mass.) set at 54° C. and the latency time to show a nociceptive response with hind paw lick or a jump was measured. If no response was observed within 30 s, the mouse was removed from the heated plate to prevent any tissue damage. Antinociception, measured as % MPE, was calculated as above.

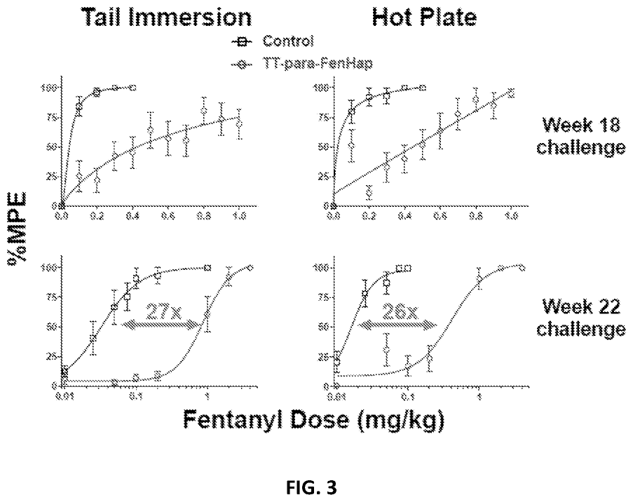

The control mice and the immunized mice were challenged with increasing doses of fentanyl at week 18 and week 22 post-vaccination. The efficacy of the fentanyl vaccine of the disclosure was assessed by measuring the maximum possible effect (% MPE) in both tail immersion and hot plate assays. The Week 18 challenge demonstrates that mice immunized with the fentanyl vaccine have a lower % MPE compared to the control mice. The Week 22 challenge demonstrates that mice immunized with the fentanyl vaccine have a 26- to 27-fold increase in ED₅₀ (50% effective dose)(FIG. 3).

### Example 13

Am-FenHap1 and Para-AmFentanylHap were also conjugated with tetanus toxoid (TT) using SM(PEG)₂, SM(PEG)₄, and SM(PEG)₈ linkers according to methods described above to yield the following:

| Fentanyl Hapten | Linker | Protein | Hapten Conjugate |
|---|---|---|---|
| Am-FenHap1 | SM(PEG)₂ | TT | TT-SM(PEG)₂-Am-FenHap1 |
| Am-FenHap1 | SM(PEG)₄ | TT | TT-SM(PEG)₄-Am-FenHap1 |
| Am-FenHap1 | SM(PEG)₈ | TT | TT-SM(PEG)₈-Am-FenHap1 |
| Para-AmFentanylHap | SM(PEG)₂ | TT | TT-SM(PEG)₂-Para-AmFentanylHap |
| Para-AmFentanylHap | SM(PEG)₄ | TT | TT-SM(PEG)₄-Para-AmFentanylHap |
| Para-AmFentanylHap | SM(PEG)₈ | TT | TT-SM(PEG)₈-Para-AmFentanylHap |

These were each subsequently mixed with ALFA (ALF43+aluminum salt) according to methods described above to yield the corresponding fentanyl vaccine formulations. A 50 μL/0.05 mL of the vaccine formulation contains TT-fentanyl hapten conjugate (10 μg), Alhydrogel (30 μg), and 3D-PHAD (20 μg).

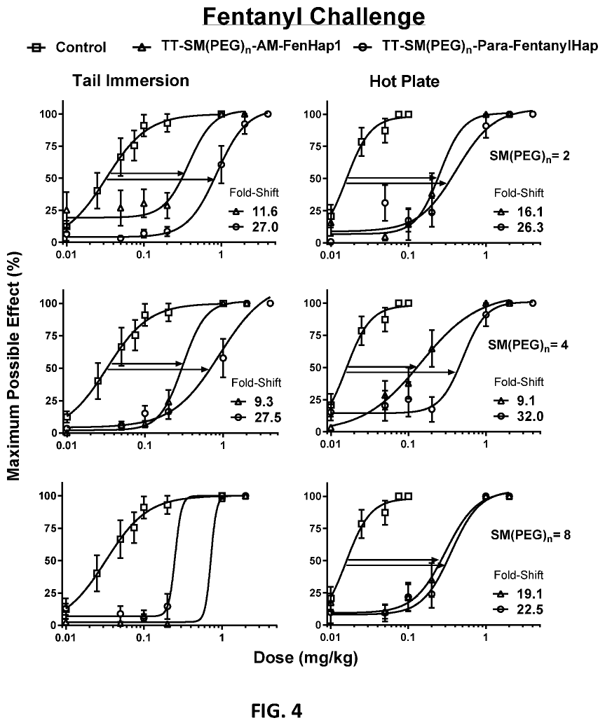

The animals were challenged by repeat-dose fentanyl by the SC route according to the methods as described above. TT-Am-FenHap1 and π-para-AmFentanylHap protected the mice against fentanyl challenge as shown in FIG. 4.

All patents and publications cited herein are incorporated by reference in their entireties.

What is claimed is:

1. A fentanyl hapten of Formula (1), (2) or (3): 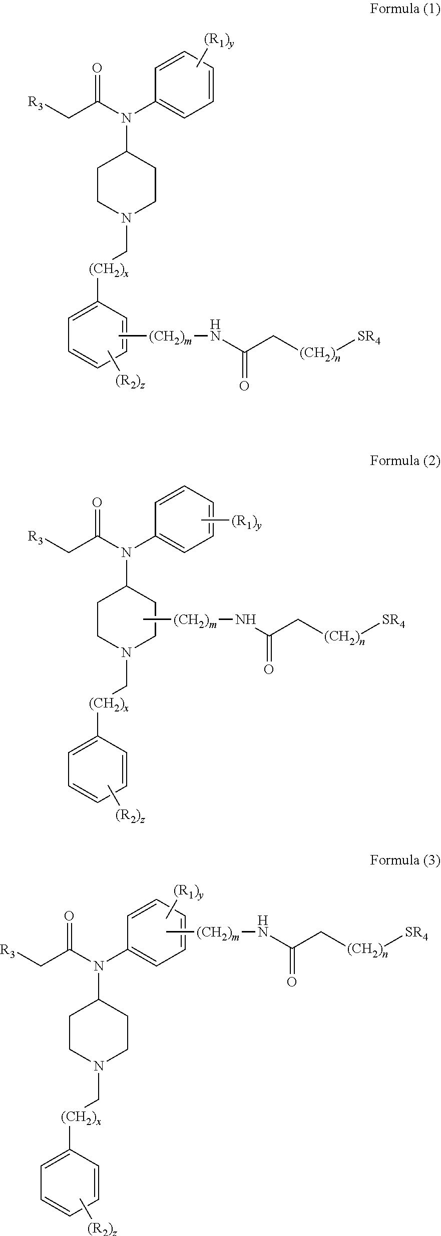
or a pharmaceutically acceptable salt of a compound of Formula (1), (2) or (3),
wherein for Formula (1):
each R₁ and R₂ is independently C₁-C₆ alkyl, C₁-C₆ alkoxy, F, Cl, Br, I, CN, NO₂, NR₅R₆, NR₅COR₆, NR₅CO₂R₇, NR₅SO₂R₇, OR₅, SR₅, SO₂R₂, SO₂NR₅R₆, COR₅, CO₂R₅, CONR₅R₆, CO₂NR₅R₆, cycloalkyl, heterocycloalkyl, aryl or heteroaryl;
R₃ is C₁-C₆ alkyl;
R₄ is H, R₅, NR₅R₆ or C(Phenyl)₃;
each R₅ is independently H or C₁-C₆ alkyl;
each R₆ is independently H or C₁-C₆ alkyl;
each R₇ is C₁-C₆ alkyl;
m=0, 1, 2, 3, 4, 5 or 6;
n=0, 1, 2, 3, 4, 5 or 6;
x=0, 1, 2, 3, 4, 5 or 6;
y=0, 1, 2, 3, 4 or 5; and
z=0, 1, 2, 3 or 4;
wherein for Formula (2):
each R₁ and R₂ is independently C₁-C₆ alkyl, C₁-C₆ alkoxy, F, Cl, Br, I, CN, NO₂, NR₅R₆, NR₅COR₆, NR₅CO₂R₇, NR₅SO₂R₇, OR₅, SR₅, SO₂R₂, SO₂NR₅R₆, COR₅, CO₂R₅, CONR₅R₆, CO₂NR₅R₆, cycloalkyl, heterocycloalkyl, aryl or heteroaryl;
R₃ is C₁-C₆ alkyl;
R₄ is H, R₅, COR₅, NR₅R₆ or C(Phenyl)₃;
each R₅ is independently H or C₁-C₆ alkyl;
each R₆ is independently H or C₁-C₆ alkyl;
each R₇ is C₁-C₆ alkyl;
m=0, 1, 2, 3, 4, 5 or 6;
n=0, 1, 2, 3, 4, 5, or 6;
x=0, 1, 2, 3, 4, 5 or 6;
y=0, 1, 2, 3, 4 or 5; and
z=0, 1, 2, 3, 4 or 5; and
wherein for Formula (3):
each R₁ and R₂ is independently C₁-C₆ alkyl, C₁-C₆ alkoxy, F, Cl, Br, I, CN, NO₂, NR₅R₆, NR₅COR₆, NR₅CO₂R₇, NR₅SO₂R₂, OR₅, SR₅, SO₂R₂, SO₂NR₅R₆, COR₅, CO₂R₅, CONR₅R₆, CO₂NR₅R₆, cycloalkyl, heterocycloalkyl, aryl or heteroaryl;
R₃ is C₁-C₆ alkyl;
R₄ is H, R₅, COR₅, NR₅R₆ or C(Phenyl)₃;
each R₅ is independently H or C₁-C₆ alkyl;
each R₆ is independently H or C₁-C₆ alkyl;
each R₇ is C₁-C₆ alkyl;
m=0, 1, 2, 3, 4, 5 or 6;
n=0, 1, 2, 3, 4, 5 or 6;
x=0, 1, 2, 3, 4, 5 or 6;
y=0, 1, 2, 3 or 4; and
z=0, 1, 2, 3, 4 or 5.

2. The fentanyl hapten according to claim 1, wherein R₃ is CH₃ for each of Formula (1), (2) and (3).

3. The fentanyl hapten according to claim 1, wherein n=1 for each of Formula (1), (2) and (3).

4. The fentanyl hapten according to claim 1, wherein x=1 for each of Formula (1), (2) and (3).

5. The fentanyl hapten according to claim 1, wherein z=0 for each of Formula (1), (2) and (3).

6. The fentanyl hapten according to claim 1, wherein y=0 for each of Formula (1), (2) and (3).

7. The fentanyl hapten according to claim 1, wherein R₄ is H for each of Formula (1), (2) and (3).

8. The fentanyl hapten according to claim 1, wherein R₃ is CH₃, x=1 and n=1 for each of Formula (1), (2) and (3).

9. The fentanyl hapten according to claim 1, wherein R₃ is CH₃, R₄ is H, x=1 and n=1 for each of Formula (1), (2) and (3).

10. The fentanyl hapten according to claim 1, wherein R₃ is CH₃, R₄ is H, x=1, y=0 and n=1 for each of Formula (1), (2) and (3).

11. The fentanyl hapten according to claim 1, wherein R₃ is CH₃, R₄ is H, x=1, y=0, z=0 and n=1 for each of Formula (1), (2) and (3).

12. The fentanyl hapten according to claim 1, wherein R₃ is CH₃, R₄ is H, x=1, y=0, z=0, m=0 and n=1 for each of Formula (1), (2) and (3).

13. The fentanyl hapten according to claim 1, wherein R₃ is CH₃, R₄ is H, x=1, y=0, z=0, m=1 and n=1 for each of Formula (1), (2) and (3).

14. The fentanyl hapten of Formula (1) or Formula (3) according to claim 1, wherein R₃ is CH₃, R₄ is H, x=1, y=0, z=0 and n=1 and the —(CH₂)ₘ—NH—CO—(CH₂)ₙ—SR₄ substituent is in the ortho position.

15. The fentanyl hapten of Formula (1) or Formula (3) according to claim 1, wherein R₃ is CH₃, R₄ is H, x=1, y=0, z=0 and n=1 and the —(CH₂)ₘ—NH—CO—(CH₂)ₙ—SR₄ substituent is in the meta position.

16. The fentanyl hapten of Formula (1) or Formula (3) according to claim 1, wherein R₃ is CH₃, R₄ is H, x=1, y=0, z=0 and n=1 and the —(CH₂)ₘ—NH—CO—(CH₂)ₙ—SR₄ substituent is in the para position.

17. An immunoconjugate comprising the fentanyl hapten of Formula (1), (2) or (3) according to claim 1.

18. An immunoconjugate comprising a carrier and the fentanyl hapten of Formula (1), (2) or (3) according to claim 1, wherein the fentanyl hapten is covalently linked to the carrier.

19. A fentanyl hapten of Formula (4), (5) or (6): 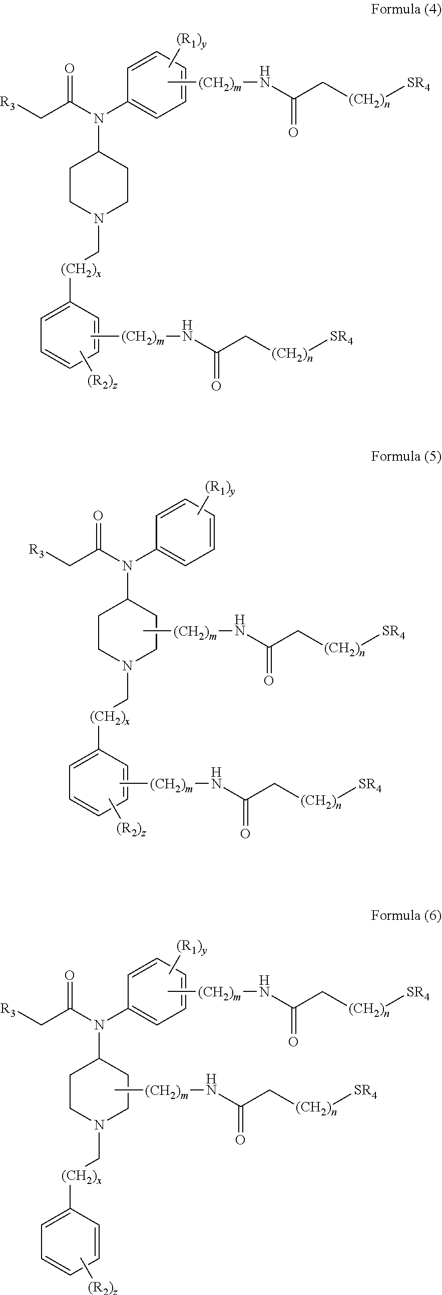
or a pharmaceutically acceptable salt of each thereof;
wherein for Formula (4):
each R₁ and R₂ is independently C₁-C₆ alkyl, C₁-C₆ alkoxy, F, Cl, Br, I, CN, NO₂, NR₅R₆, NR₅COR₆, NR₅CO₂R₇, NR₅SO₂R₇, OR₅, SR₅, SO₂R₂, SO₂NR₅R₆, COR₅, CO₂R₅, CONR₅R₆, CO₂NR₅R₆, cycloalkyl, heterocycloalkyl, aryl or heteroaryl;
R₃ is C₁-C₆ alkyl;
each R₄ is independently H, R₅, COR₅, NR₅R₆ or C(Phenyl)₃;
each R₅ is independently H or C₁-C₆ alkyl;
each R₆ is independently H or C₁-C₆ alkyl;
each R₇ is C₁-C₆ alkyl;
m=0, 1, 2, 3, 4, 5 or 6;
n=0, 1, 2, 3, 4, 5, or 6;
x=0, 1, 2, 3, 4, 5 or 6;
y=0, 1, 2, 3 or 4; and
z=0, 1, 2, 3 or 4;
wherein for Formula (5):
each R₁ and R₂ is independently C₁-C₆ alkyl, C₁-C₆ alkoxy, F, Cl, Br, I, CN, NO₂, NR₅R₆, NR₅COR₆, NR₅CO₂R₂, NR₅SO₂R₂, OR₅, SR₅, SO₂R₇, SO₂NR₅R₆, COR₅, CO₂R₅, CONR₅R₆, CO₂NR₅R₆, cycloalkyl, heterocycloalkyl, aryl or heteroaryl;
R₃ is C₁-C₆ alkyl;
each R₄ is independently H, R₅, COR₅, NR₅R₆ or C(Phenyl)₃;
each R₅ is independently H or C₁-C₆ alkyl;
each R₆ is independently H or C₁-C₆ alkyl;
each R₇ is C₁-C₆ alkyl;
m=0, 1, 2, 3, 4, 5 or 6;
n=0, 1, 2, 3, 4, 5, or 6;
x=0, 1, 2, 3, 4, 5 or 6;
y=0, 1, 2, 3, 4 or 5; and
z=0, 1, 2, 3 or 4; and
wherein for Formula (6):
each R₁ and R₂ is independently C₁-C₆ alkyl, C₁-C₆ alkoxy, F, Cl, Br, I, CN, NO₂, NR₅R₆, NR₅COR₆, NR₅CO₂R₇, NR₅SO₂R₇, OR₅, SR₅, SO₂R₂, SO₂NR₅R₆, COR₅, CO₂R₅, CONR₅R₆, CO₂NR₅R₆, cycloalkyl, heterocycloalkyl, aryl or heteroaryl;
R₃ is C₁-C₆ alkyl;
each R₄ is independently H, R₅, COR₅, NR₅R₆ or C(Phenyl)₃;
each R₅ is independently H or C₁-C₆ alkyl;
each R₆ is independently H or C₁-C₆ alkyl;
each R₇ is C₁-C₆ alkyl;
m=0, 1, 2, 3, 4, 5 or 6;
n=0, 1, 2, 3, 4, 5, or 6;
x=0, 1, 2, 3, 4, 5 or 6;
y=0, 1, 2, 3 or 4; and
z=0, 1, 2, 3, 4 or 5.

20. The fentanyl hapten according to claim 19, wherein R₃ is CH₃ for each of Formula (4), (5) and (6).

21. The fentanyl hapten according to any one of claim 19 or 20, wherein each R₄ is independently H or C(Phenyl)₃ for each of Formula (4), (5) and (6).

22. An immunoconjugate comprising the fentanyl hapten of Formula (4), (5) or (6) according to any one of claims 19-21.

23. An immunoconjugate comprising a carrier and the fentanyl hapten of Formula (4), (5) or (6) according to any one of claims 19-21, wherein the fentanyl hapten is covalently linked to the carrier.

24. The immunoconjugate according to any one of claims 17-18 and 22-23, wherein the carrier is selected from the group consisting of tetanus toxoid, CRM197, diphtheria toxoid, recombinant deactivated tetanus toxoid, recombinant tetanus A chain, recombinant tetanus B chain, exotoxin A, Keyhole limpet hemocyanin (KLH) and recombinant KLH.

25. A composition comprising an immunologically effective amount of the immunoconjugate according to claim 18 or claim 23 and a physiologically acceptable vehicle.

26. The composition according to claim 25, further comprising an adjuvant.

27. The composition according to claim 26, wherein the adjuvant is selected from the group consisting of an ALF liposome, ALFA, ALFQ, ALFQA, aluminium hydroxide, aluminium phosphate, alum and monophosphoryl lipid A containing adjuvants.

28. The composition according to claim 27, wherein the immunoconjugate is embedded, associated with or attached to the adjuvant.

29. A method for inducing an anti-fentanyl immune response in a subject, comprising immunizing the subject with an immunologically effective amount of the composition according to claim 25.

30. The method according to claim 29, wherein the carrier is tetanus toxoid.

31. A method for inducing an anti-fentanyl immune response without inducing an immune response to a carrier moiety in a subject, comprising immunizing the subject with an immunologically effective amount of the composition according to claim 25.

32. A vaccine composition comprising the immunoconjugate according to claim 18 or claim 23.

33. The vaccine composition according to claim 32, further comprising an adjuvant.

34. The vaccine composition according to claim 33, wherein the adjuvant is selected from the group consisting of an ALF liposome, ALFA, ALFQ, ALFQA, aluminium hydroxide, aluminium phosphate, alum and monophosphoryl lipid A containing adjuvants.

35. The immunoconjugate according to claim 18 or claim 23, wherein the fentanyl hapten is covalently linked to the carrier through a linking moiety, where the linking moiety is a NHS-maleimide crosslinker.

36. A composition comprising an immunologically effective amount of the immunoconjugate according to claim 17 or claim 22 and a physiologically acceptable vehicle.

37. The composition according to claim 36, further comprising an adjuvant.

38. The composition according to claim 37, wherein the adjuvant is selected from the group consisting of an ALF liposome, ALFA, ALFQ, ALFQA, aluminium hydroxide, aluminium phosphate, alum and monophosphoryl lipid A containing adjuvants.

39. The composition according to claim 37, wherein the immunoconjugate is embedded, associated with or attached to the adjuvant.

40. A method for inducing an anti-fentanyl immune response in a subject, comprising immunizing the subject with an immunologically effective amount of the composition according to claim 36.

41. The method according to claim 40, wherein the immunoconjugate comprises a carrier, wherein the carrier is tetanus toxoid.

42. A method for inducing an anti-fentanyl immune response without inducing an immune response to a carrier moiety in a subject, comprising immunizing the subject with an immunologically effective amount of the composition according to claim 36.

43. A vaccine composition comprising the immunoconjugate according to claim 17 or claim 22.
